(19) United States
(12) Patent Application Publication
(10) Pub. No.: US 2026/0097233 A1
(43) Pub. Date: Apr. 9, 2026

(54) TISSUE ISOLATION DEVICES CAPABLE OF REPEATED INFLATION AND DEFLATION IN VIVO

(71) Applicant: BEIJING QIFAN BOCHUANG MEDICAL TECHNOLOGY CO., LTD., Beijing (CN)

(72) Inventors: Qutong XI, Beijing (CN); Xin LIANG, Beijing (CN); Xiaopeng LIU, Beijing (CN)

(73) Assignee: BEIJING QIFAN BOCHUANG MEDICAL TECHNOLOGY CO., LTD., Beijing (CN)

(21) Appl. No.: 19/417,896

(22) Filed: Dec. 12, 2025

Related U.S. Application Data
(63) Continuation-in-part of application No. PCT/CN2024/097030, filed on Jun. 3, 2024.

(30) Foreign Application Priority Data
Jun. 12, 2023 (CN) 202310688295.5
Aug. 1, 2025 (CN) 202511076745.0

(51) Int. Cl. A61N 5/10; A61M 19/00; A61M 37/00

(52) U.S. Cl. CPC A61N 5/10; A61M 19/00; A61M 37/00; A61M 2202/0007; A61M 2202/048; A61M 2202/049; A61M 2205/0238; A61N 2005/1094; A61N 2005/1096

(57) ABSTRACT

The present disclosure relates to a tissue isolation device capable of repeated inflation and deflation in vivo. The device includes: an isolation balloon arranged between healthy tissues and target tissues for a radiotherapy course, and the isolation balloon is removed from a body of a subject following the radiotherapy course. The isolation balloon is operable to assume an inflated state and a deflated state, the isolation balloon in the inflated state is used to isolate the healthy tissues and the target tissues. The isolation balloon includes a first contact surface and a second contact surface, under the inflated state, the first contact surface contacts the healthy tissues, and the second contact surface contacts the target tissues, a distance between the first contact surface and the second contact surface is in a preset range.

### CROSS-REFERENCE TO RELATED APPLICATIONS

The application is a Continuation-in-part of International application No. PCT/CN2024/097030, filed on Jun. 3, 2024, which claims priority to Chinese Patent Application No. 202310688295.5, filed on Jun. 12, 2023, and Chinese Patent Application No. 202511076745.0, filed on Aug. 1, 2025, the entire contents of each of which are hereby incorporated by reference.

### TECHNICAL FIELD

The present disclosure generally relates to a field of interventional radiation oncology, and in particular to a tissue isolation device capable of repeated inflation and deflation in vivo.

### BACKGROUND

Tumor radiotherapy is a local therapeutic modality that utilizes radiation to treat tumors. The radiation mainly includes high-energy X-rays, electron beams, and proton beams generated by an accelerator. During tumor radiotherapy, while killing tumor cells, the radiation inevitably damages surrounding healthy tissues and may even cause serious irreversible complications, such as radiation proctitis, rectal perforation, or the like.

The prior art CN202110281992 discloses a prostate and rectum isolation device, which provides an elliptical isolation instrument and controls inflation or fluid filling by means of a spring-type control valve. However, tumor radiotherapy is a long-cycle treatment, not a single treatment; a radiotherapy course typically includes 20-40 irradiation treatments, with one irradiation per day (approximately 10-20 minutes), and a course needs to last for 4-8 weeks. This technology fails to account for the radiotherapy cycle. The inflation/fluid filling valve is designed to be located outside the body, making it impossible for patients to carry it long-term, and the feasibility of translation into clinical practice is relatively low. In addition, the elliptical configuration is prone to causing positional movement during tissue isolation.

In recent years, European and American countries have adopted percutaneous injection of degradable hydrogel or hyaluronic acid to form a physical isolation between the prostate and the rectum to reduce the radiation dose to the rectum. However, the degradation cycle of such materials in vivo typically lasts for 3-6 months. During this period, the injected isolation hydrogel or hyaluronic acid continuously compresses the intestine, posing risks of intestinal perforation and constipation to the patient. Moreover, after radiotherapy, it continues to compress the intestine, increasing the risk of long-term complications such as intestinal perforation and constipation, and it cannot dynamically adjust the isolation state according to treatment needs.

Therefore, an objective is to provide a tissue isolation device capable of repeated inflation and deflation in vivo to avoid continuous compression of internal tissues and reduce related complications.

### SUMMARY

One or more embodiments of the present disclosure provide a tissue isolation device capable of repeated inflation and deflation in vivo, comprising: an isolation balloon, wherein the isolation balloon is arranged between healthy tissues and target tissues for a radiotherapy course, and the isolation balloon is removed from a body of a subject upon completion of the radiotherapy course; the isolation balloon is operable to assume an inflated state and a deflated state, the isolation balloon in the inflated state is used to isolate the healthy tissues and the target tissues, and the isolation balloon in the deflated state is used to maintain a physiological state of the healthy tissues and the target tissues; the isolation balloon includes a first contact surface and a second contact surface, in the inflated state, the first contact surface is in contact with the healthy tissues, and the second contact surface is in contact with the target tissues; and when the isolation balloon is in the inflated state, a minimum distance between the first contact surface and the second contact surface is greater than or equal to a distance required for a radiation dose to fall off to within a tolerance threshold of the healthy tissues, a maximum distance between the first contact surface and the second contact surface is less than or equal to a maximum tolerable distraction distance of the healthy tissues.

### BRIEF DESCRIPTION OF THE DRAWINGS

The present disclosure will be further illustrated by way of exemplary embodiments. The exemplary embodiments will be described in detail with reference to the accompanying drawings. The embodiments are not limiting. In the embodiments, the same reference numerals denote the same structures, wherein:

FIG. 1 is a schematic diagram illustrating a top view of a structure of a tissue isolation device when a connection tube is arranged at a side of an implant abutment and perpendicular to the implant abutment according to some embodiments of the present disclosure;

FIG. 2 is a schematic diagram illustrating a bottom view of a structure of a tissue isolation device when a connection tube is arranged at a side of an implant abutment and perpendicular to an implant abutment according to some embodiments of the present disclosure;

FIG. 3A is a schematic diagram illustrating a cross-sectional structure of a front view plane of an isolation balloon according to some embodiments of the present disclosure;

FIG. 3B is a schematic diagram illustrating a cross-sectional structure of a top view plane of an isolation balloon according to some embodiments of the present disclosure;

FIG. 3C is a schematic diagram illustrating a cross-sectional structure of a right view plane of an isolation balloon according to some embodiments of the present disclosure;

FIG. 4 is a schematic diagram illustrating a cross-sectional structure of a bottom plane of an isolation balloon and an internal connection tube according to some embodiments of the present disclosure;

FIG. 5 is a schematic diagram illustrating a structure of an isolation balloon and connection tube with a supporting and positioning rod without an implant abutment according to some embodiments of the present disclosure;

FIG. 6 is a schematic diagram illustrating a partial longitudinal cross-sectional structure of an inflation connection structure according to some embodiments of the present disclosure;

FIG. 7 is a schematic diagram illustrating a partial longitudinal cross-sectional structure of an inflation connection structure with a closure head according to some embodiments of the present disclosure;

FIG. 8 is a schematic diagram illustrating a bottom plane of an implant abutment when a connection tube is arranged below the implant abutment according to some embodiments of the present disclosure;

FIG. 9 is a schematic diagram illustrating a connection structure between an implant abutment and a connection tube when the connection tube is arranged below the implant abutment according to some embodiments of the present disclosure;

FIG. 10 is a schematic diagram illustrating a transverse cross-sectional structure of a connection tube provided with an inflation lumen and a positioning lumen according to some embodiments of the present disclosure;

FIG. 11 is a schematic diagram illustrating an overall structure of a connection tube provided with an inflation lumen and a positioning lumen according to some embodiments of the present disclosure;

FIG. 12 is a schematic diagram illustrating a structure of a heating element and a cooling element according to some embodiments of the present disclosure;

FIG. 13 is a schematic diagram illustrating a specific implementation in which an isolation balloon is arranged between deep tumor tissues and healthy tissues according to some embodiments of the present disclosure; wherein a connection tube is arranged below the implant abutment;

FIG. 14 is a schematic diagram illustrating a specific implementation in which an isolation balloon is arranged between prostate and rectum according to some embodiments of the present disclosure; wherein a connection tube is arranged at a side of an implant abutment and perpendicular to a bottom of the implant abutment;

FIG. 15 is a schematic diagram illustrating an overall structure of a tissue isolation device according to some embodiments of the present disclosure;

FIG. 16 is a schematic diagram illustrating a cross-sectional structure in which a portion of the connection tube and a fluid-filling component separated from the connection tube according to some embodiments of the present disclosure;

FIG. 17 is a schematic diagram illustrating a structure of a fluid-filling component according to some embodiments of the present disclosure;

FIG. 18 is a schematic diagram illustrating a structure of a fluid-filling structure according to other embodiments of the present disclosure;

FIG. 19 is a schematic diagram illustrating a cross-sectional structure of a fluid-filling component with an occlusion valve as a sealing structure according to some embodiments of the present disclosure;

FIG. 20 is a schematic diagram illustrating a structure in which a fluid-filling component is separated from an isolation balloon according to some embodiments of the present disclosure;

FIG. 21 is a schematic diagram illustrating a cross-sectional structure of a fluid-filling component according to some embodiments of the present disclosure.

Reference numerals: 1, isolation balloon; 11, first contact surface; 12, second contact surface; 13, extension tube; 2, connection tube; 21, inflation lumen; 211, inflation through hole; 22, positioning lumen; 221, closure cap; 222, positioning port; 3, inflation connection structure; 4, supporting and positioning rod; 51, implant abutment; 52, communicated cavity; 53, seal structure; 54, protective baffle; 541, through hole; 55, communicated port; 56, suture ring; 61, fluid-filling component; 611, fluid-filling structure; 6111, port body; 61111, cavity; 61112, first portion; 61113, second portion; 61121, resilient septum; 61122, occlusion valve; 6113, connection head; 6114, closure head; 612, fixing tab; 6121, adhesive layer; 6122, plurality of suture holes; 71, heating element; 72, cooling element.

### DETAILED DESCRIPTION

To more clearly illustrate the technical solutions of the embodiments of the present disclosure, the accompanying drawings used in the description of the embodiments will be briefly introduced below. Obviously, the accompanying drawings in the following description are merely some examples or embodiments of the present disclosure. For those of ordinary skill in the art, without creative effort, the present disclosure can be applied to other similar scenarios based on these accompanying drawings. Unless obviously obtained from the context or the context illustrates otherwise, the same numeral in the drawings refers to the same structure or operation.

It should be understood that the terms “system”, “device”, “unit” and/or “module” used herein are methods for distinguishing components, elements, parts, sections, or assemblies of different levels. However, if other words can achieve the same purpose, the words can be replaced by other expressions.

As shown in the present disclosure and the claims, unless the context clearly indicates an exception, the terms “a”, “an”, “one” and/or “the” are not limited to the singular, and can also include the plural. Generally, the terms “include” and “comprise” only indicate that clearly identified steps and elements are included, and these steps and elements do not constitute an exclusive list. A method or device may also include other steps or elements.

Distance protection is still the most important and effective radiological protection measure. Modern radiotherapy instruments have high precision, isolating a distance of 1-2 cm between healthy tissues and tumor tissues can significantly reduce the radiation dose and greatly mitigate the risk of complications. Therefore, a tissue isolation device that can be repeatedly inflated and deflated in vivo is provided. An isolation balloon with a preset shape can form an effective and stable isolation distance between the healthy tissues and the target tissues. A cooperation of a soft connection tube, an inflation connection structure implanted in subcutaneous tissues, and the isolation balloon can achieve isolation during daily radiotherapy and non-isolation when radiotherapy is not performed. It can not only be used for rectal isolation during prostate cancer radiotherapy, but also for isolation of adjacent healthy tissues during radiotherapy for various other tumors.

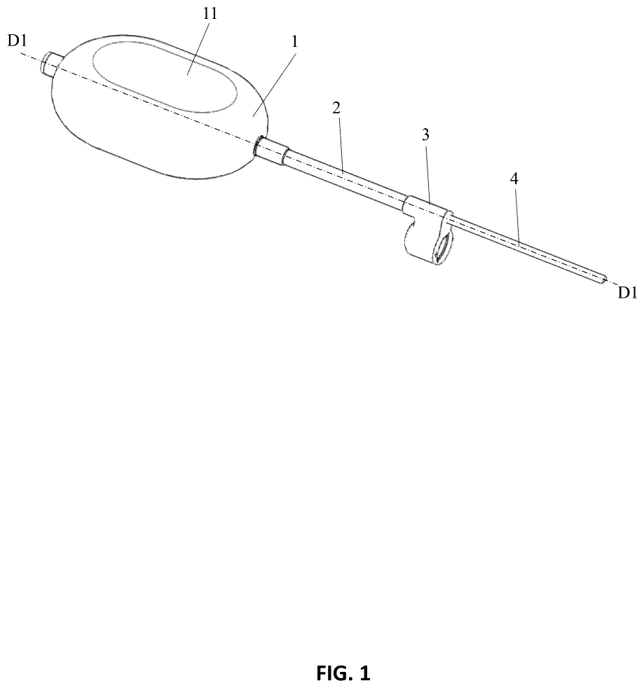

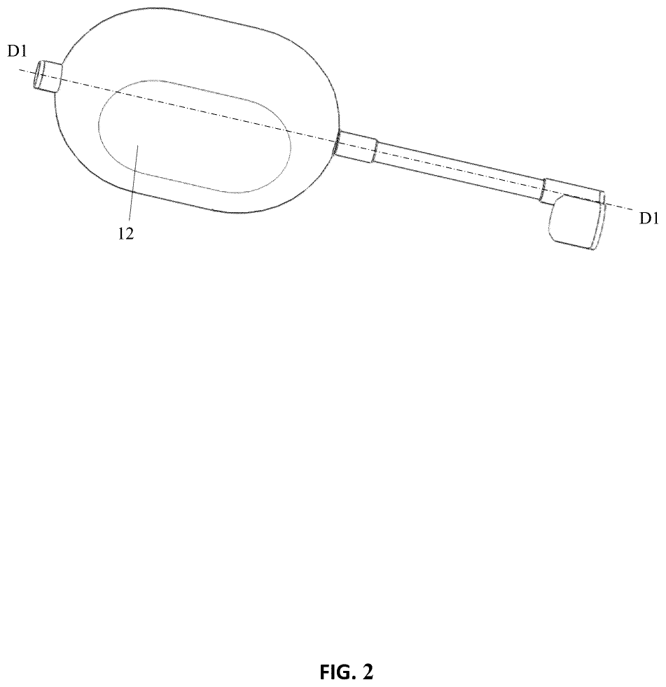

Some embodiments of the present disclosure provide a tissue isolation device that can be repeatedly inflated and deflated in vivo. FIG. 1 is a schematic diagram illustrating a top view of a structure of a tissue isolation device when a connection tube is arranged at a side of an implant abutment and perpendicular to the implant abutment according to some embodiments of the present disclosure. FIG. 2 is a schematic diagram illustrating a bottom view of a structure of a tissue isolation device when a connection tube is arranged at a side of an implant abutment and perpendicular to an implant abutment according to some embodiments of the present disclosure.

In some embodiments, as shown in FIG. 1-FIG. 2, the tissue isolation device includes: an isolation balloon 1, wherein the isolation balloon 1 is arranged between healthy tissues and target tissues for a radiotherapy course, and the isolation balloon 1 is removed from the subject's body upon completion of the radiotherapy course. The isolation balloon 1 is operable to assume an inflated state and a deflated state, the isolation balloon 1 in the inflated state is used to isolate the healthy tissues and the target tissues, and the isolation balloon 1 in the deflated state is used to maintain a physiological state of the healthy tissues and the target tissues. The isolation balloon 1 includes a first contact surface 11 and a second contact surface 12, in the inflated state, the first contact surface 11 is in contact with the healthy tissues, and the second contact surface 12 is in contact with the target tissues.

The isolation balloon 1 is configured to isolate the healthy tissues from the target tissues during a radiotherapy process, thereby reducing a radiation dose received by the healthy tissues.

The healthy tissues refer to normal organs and tissues adjacent to a tumor, e.g., intestines, glands, lymph, or the like. The target tissues refer to tissues where the tumor is located and require radiotherapy.

In some embodiments, the isolation balloon 1 adopts a double-layer membrane structure. For example, an inner layer of the isolation balloon 1 is a medical-grade silicone or polyurethane membrane that is leak-proof, and an outer layer is an anti-adhesion coating (e.g., a polyethylene glycol-modified surface), thereby reducing adhesion to surrounding tissues. In some embodiments, a shape of the isolation balloon 1 may be set according to requirements. For example, the isolation balloon 1 is a flattened sac shape, a dumbbell shape, or a customized curved surface, or the like, thereby adapting to different radiotherapy sites (e.g., a prostate-rectum space, a breast-chest wall space, etc.).

The inflated state refers to a state where the isolation balloon 1 is inflated to form an effective isolation space inside. The isolation balloon 1 in the inflated state may isolate the healthy tissues from the target tissues. The deflated state refers to a state where the isolation balloon 1 is emptied and contracted. The setting of repeated inflation and deflation is crucial when the tumor is located in a site with a limited space, or when surrounding tissues cannot tolerate long-term compression. The isolation balloon 1 may be inflated to the inflated state by percutaneous puncture before each treatment, and the inflation medium may be evacuated to retract the isolation balloon 1 to the deflated state after the treatment, thereby minimizing compression and injury to the healthy tissues and reducing damage to the tissues during radiotherapy.

In some embodiments, a shape of the first contact surface 11 is adapted to an anatomical structure of the healthy tissues, and a shape of the second contact surface 12 may be adjusted according to a radiotherapy target area. For example, in prostate cancer radiotherapy, the first contact surface 11 conforms to a rectal wall and is designed as an arc to avoid compression damage; and the second contact surface 12 conforms to a prostate, and a shape of the second contact surface 12 matches a tumor target area. As another example, in breast cancer radiotherapy, the first contact surface 11 conforms to a chest wall or lung tissue, the second contact surface 12 conforms to a postoperative tumor bed, and a concave structure may be customized to fix a position.

In some embodiments, when the isolation balloon 1 is in the inflated state, a minimum distance between the first contact surface 11 and the second contact surface 12 is greater than or equal to a distance required for a radiation dose to fall off to within a tolerance threshold of the healthy tissues, and a maximum distance between the first contact surface 11 and the second contact surface 12 is less than or equal to a maximum tolerable distraction distance of the healthy tissues. When the two distances (the minimum distance and the maximum distance) conflict, the maximum distance not exceeding the maximum distance that the stretched tissues may withstand shall prevail. The above setting can avoid irreversible damage to a physiological state of the tissues while reducing a radiotherapy radiation dose.

The minimum distance refers to a minimum value of distances along a radial direction of the isolation balloon between the first contact surface 11 and the second contact surface 12. The maximum distance refers to a maximum value of distances along the radial direction of the balloon between the first contact surface 11 and the second contact surface 12. The radial direction of the isolation balloon 1 refers to a direction perpendicular to an axial direction of the isolation balloon 1. The axial direction of the isolation balloon 1 may be a central axis direction of the isolation balloon 1, e.g., an axis D1-D1 direction as shown in FIG. 1-FIG. 2.

In some embodiments, the first contact surface 11 and the second contact surface 12 are the same plane, and orthogonal projections of the first contact surface 11 and the second contact surface 12 on a horizontal plane overlap, thereby ensuring stability of an overall setting state.

In some embodiments, the first contact surface 11 and the second contact surface 12 are two planes arranged in parallel, thereby effectively maintaining a stable distance with a consistent overall distance.

In some embodiments, the first contact surface 11 and the second contact surface 12 are curved surfaces that are low in the middle and high on both sides.

In some embodiments, outer sides or peripheral regions of the first contact surface 11 and the second contact surface 12 are higher than (a central region of) the first contact surface 11 and (a central region of) the second contact surface 12, thereby ensuring that the isolation balloon 1 forms a structure that is low in the middle and high on both sides to confine the tissues within a formed space, thereby avoiding positional movement of the tissues during the radiotherapy process.

In some embodiments, on a surface of the isolation balloon 1, the outer sides or the peripheral regions of the first contact surface 11 and the second contact surface 12 are lower than the first contact surface 11 and the second contact surface 12, and the peripheral regions of the first contact surface 11 and the second contact surface 12 blend through a radial contour, i.e., the first contact surface 11 and the second contact surface 12 are connected by an arc transition, thereby ensuring that the isolation balloon 1 does not cause unnecessary isolation pressure to surrounding tissues and has better protection for the tissues.

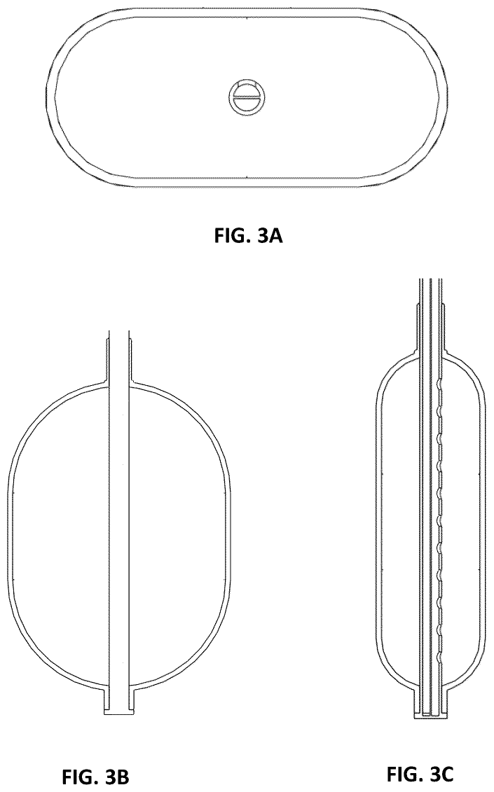

FIG. 3A is a schematic diagram illustrating a cross-sectional structure of a front view plane of an isolation balloon according to some embodiments of the present disclosure. FIG. 3B is a schematic diagram illustrating a cross-sectional structure of a top view plane of an isolation balloon according to some embodiments of the present disclosure. FIG. 3C is a schematic diagram illustrating a cross-sectional structure of a right view plane of an isolation balloon according to some embodiments of the present disclosure.

In some embodiments, as shown in FIG. 3A, FIG. 3B, and FIG. 3C, a shape of the first contact surface 11 and a shape of the second contact surface 12 may be set as an elliptical shape or a notch shape, and an overall top view shape of the isolation balloon 1 is a notch shape, a side view shape is a notch shape, and a front view shape is also a notch shape.

In some embodiments, in the inflated state, a distance between the first contact surface 11 and the second contact surface 12 ranges from 1 cm to 2 cm. The distance meets the isolation needs of normal tissues during radiotherapy for most tumors currently, satisfies the requirements of reducing the radiation dose to a level tolerable by the healthy tissues, and avoids traction injury to the tissues due to an excessively large isolation distance.

In some embodiments, materials of balloon walls corresponding to the first contact surface 11 and the second contact surface 12 may be different from or the same as materials of other parts of the isolation balloon 1. For example, the balloon walls of the first contact surface 11 and the second contact surface 12 are made of non-elastic materials. As another example, the entire isolation balloon 1 is made of non-elastic soft material, so that a planar shape is easily formed and the distance between the first contact surface 11 and the second contact surface 12 is maintained effectively.

In some embodiments, the isolation balloon 1 may be integrally formed, such as by blow molding in a mold, thereby ensuring the simplicity of the preparation method of the isolation balloon 1.

In some embodiments, the isolation balloon 1 is coated with a drug-carrying coating. The first contact surface 11 and the second contact surface 12 are respectively coated with a drug-carrying coating, and the first contact surface 11 and the second contact surface 12 carry different drugs. The first contact surface 11 is loaded with a radioprotective agent and/or a slow-release local anesthetic. The second contact surface 12 is loaded with a chemotherapeutic agent and/or the slow-release local anesthetic.

The drug-carrying coating refers a functional coating layer that is capable of carrying drugs. In some embodiments, the drug-carrying coating may carry the drugs by physical adsorption, chemical bonding, microsphere embedding, etc., thereby achieving controlled release of the drug.

In some embodiments of the present disclosure, accurately setting the radioprotective agent and the slow-release local anesthetic drug on the first contact surface 11 can effectively alleviate pain and reduce interference of radiotherapy on healthy tissues. Accurately setting the chemotherapeutic agent and the slow-release local anesthetic drug on the second contact surface 12 can effectively alleviate pain of target tissues and perform targeted chemotherapy. The therapeutic effect is improved by the dual treatment method.

In some embodiments, for superficial tissues, the isolation balloon 1 is set between the healthy tissues and the target tissues by puncture under B-ultrasound or CT guidance. For deep tissues, the isolation balloon 1 is set between the healthy tissues and the target tissues by endoscopic surgery.

Whether for isolation of the superficial tissues or the deep tissues, implantation is performed through minimally invasive small incision surgery by puncture or endoscopy. The entire implantation process does not require open surgery, and the entire manner of placing the isolation balloon is fast and minimally invasive.

Some embodiments of the present disclosure provide beneficial effects which include but are not limited to the following content. (1) The isolation balloon 1 is set to be repeatedly inflated and deflated during the entire radiotherapy course; inflation is only implemented during radiotherapy, and deflation is implemented after radiotherapy ends, avoiding continuous tissue compression and damage caused by continuous isolation; and inflation is performed before treatment every day, fluid drainage is performed after treatment, and inflation lasts only 10-20 minutes every day, thereby minimizing the tissue compression. (2) A stable isolation distance is established by limiting the distance between the first contact surface 11 and the second contact surface 12 in the inflated state; an effective distance is established once inflation is performed, without a cumbersome operation process. (3) After a radiotherapy course ends, the isolation balloon 1 can be quickly withdrawn by deflating the substance in the isolation balloon 1 and preferably forming a negative pressure; and there is no residue in the body of a target subject, tissue compression and a foreign body sensation are completely relieved.

It should be noted that the above description of the tissue isolation device is only for convenience of description and cannot limit the present disclosure to the scope of the embodiments described. It can be understood that for those skilled in the art, after understanding the principle of the tissue isolation device, various components may be arbitrarily combined, or sub-components may be formed and connected to other components without departing from this principle.

In some embodiments, as shown in FIG. 1, the tissue isolation device further includes an inflation connection structure 3. The inflation connection structure 3 is configured to connect the isolation balloon 1 and a filling structure located outside the body of the target subject. The inflation connection structure 3 is implanted in subcutaneous tissues of the target subject, and the inflation connection structure 3 and the filling structure are connected to control inflation and deflation of the isolation balloon 1.

For example, before a radiotherapy session, the inflation connection structure 3 communicates with the filling structure to complete inflation of the isolation balloon 1 and maintain the inflated state. After the radiotherapy session ends, the inflation connection structure 3 communicates with the filling structure to complete deflation of the isolation balloon 1 and maintain the original position.

In some embodiments, the inflation connection structure 3 may be designed as a one-way valve or a sealable interface form. For example, the inflation connection structure 3 may include a subcutaneously implanted infusion port, a catheter system with an anti-reflux valve, or the like.

In some embodiments, the inflation connection structure 3 may be implanted and fixed subcutaneously after the isolation balloon 1 is implanted.

The target subject refers to a patient who needs radiotherapy treatment.

The filling structure is used to inject a filling medium into the isolation balloon 1. In some embodiments, the filling structure may include a non-invasive puncture needle, and a tail end of the non-invasive puncture needle may be connected to a manual syringe, a liquid filling pump, etc. The filling structure may also be other structures used for injecting the filling medium.

The non-invasive puncture needle refers to a blunt or side-hole puncture needle specifically used for piercing a seal structure 53.

In some embodiments, the filling medium may include a gas or a liquid. For example, the filling medium may be a mixed liquid of physiological saline and a contrast agent.

In some embodiments, during a treatment session, the filling structure may not be removed and remains in communication with the inflation connection structure 3 until the treatment ends and the isolation balloon 1 is deflated, thereby reducing the impact of repeated punctures on the patient and on the repeated piercing of the seal structure 53.

In some embodiments of the present disclosure, the inflation connection structure 3 is implanted and fixed subcutaneously to enable the entire device to be completely implanted in the body and carried for a long term. The patient's daily life (e.g., bathing) is unaffected, and the risk of infection is low. The inflation connection structure 3 cooperates with the filling structure to flexibly control the state of the isolation balloon 1.

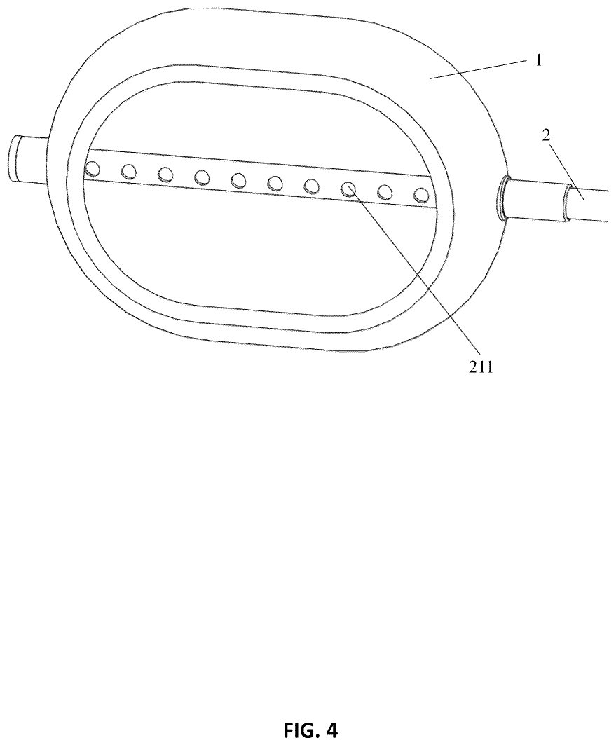

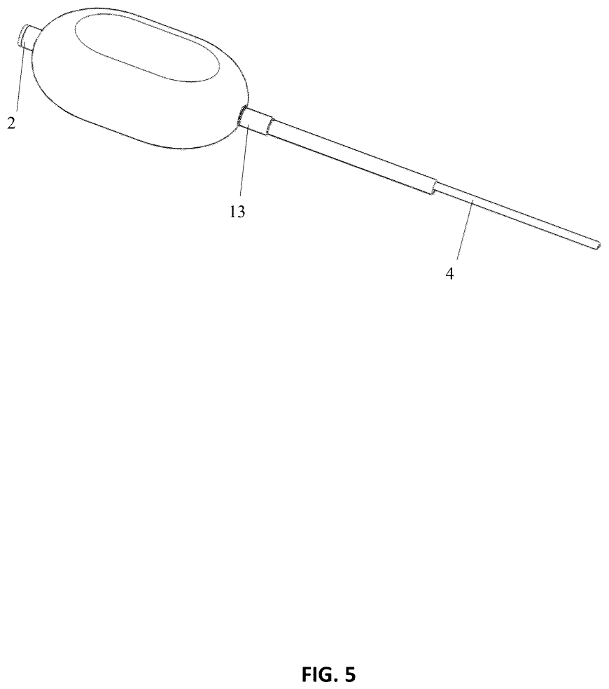

FIG. 4 is a schematic diagram illustrating a cross-sectional structure of a bottom plane of an isolation balloon and an internal connection tube according to some embodiments of the present disclosure. FIG. 5 is a schematic diagram illustrating a structure of an isolation balloon and connection tube with a supporting and positioning rod without an implant abutment according to some embodiments of the present disclosure.

In some embodiments, as shown in FIG. 1 and FIG. 4-FIG. 5, the tissue isolation device further includes a connection tube 2. The connection tube 2 is configured to communicate the inflation connection structure 3 and the isolation balloon 1. An end of the connection tube 2 that first enters the body of the target subject is designated as a first end, and the first end of the connection tube 2 is arranged at least partially into an interior of the isolation balloon 1. The first end of the connection tube 2 is arranged with an inflation through hole 211 communicated with the isolation balloon 1.

In some embodiments, an end of the isolation balloon 1 that first enters the body of the target subject is a first end, and an end that later enters the body of the target subject is a second end. The first end of the connection tube 2 extends inward from the second end of the isolation balloon 1 and extends outward from the first end of the isolation balloon 1.

The connection tube 2 is used to provide a channel for the filling medium to be injected into or discharged from the isolation balloon 1. In some embodiments, the connection tube 2 may be a soft connection tube, such as a medical silicone connection tube, etc. In some embodiments, the connection tube 2 may be made of other medical polymer materials, such as polyether block amide (Pebax), polytetrafluoroethylene (PTFE), polyamide material (PA), polyurethane (PU), etc. The setting can ensure the safety of the connection tube 2 being set in the body of the target subject for a long time, and avoid damage to tissues by the connection tube 2.

In some embodiments, a count of the inflation through hole 211 may be one or more. The inflation through hole 211 is located at a portion of the connection tube 2 extending into the isolation balloon 1 (i.e., the first end of the connection tube 2). The filling medium in the connection tube 2 may be released from the inflation through hole 211 into an interior of the isolation balloon 1.

In some embodiments, as shown in FIG. 5, both ends of the isolation balloon 1 are provided with extension tubes 13, and the extension tubes 13 are sealingly connected to the connection tube 2. The sealing connection may be implemented by biological glue adhesion, etc.

In some embodiments of the present disclosure, it can be ensured that the connection tube 2 is stably placed in the body of the target subject for a long time by setting the connection tube 2 via the above manner, thereby avoiding damage to internal tissues caused by patient activity to enhance the safety. The first end of the connection tube 2 is extended into the interior of the isolation balloon 1 and is provided with the inflation through hole 211, so that the filling medium is directly and stably injected into the isolation balloon 1, effectively preventing leakage at the connection and filling failure.

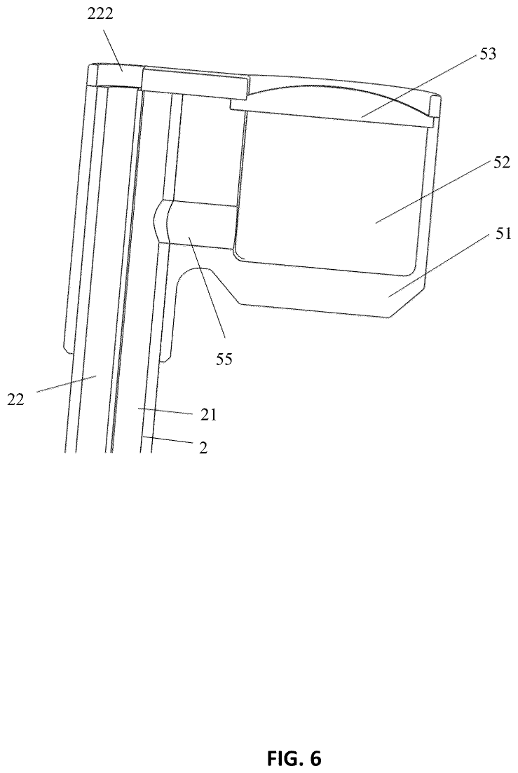

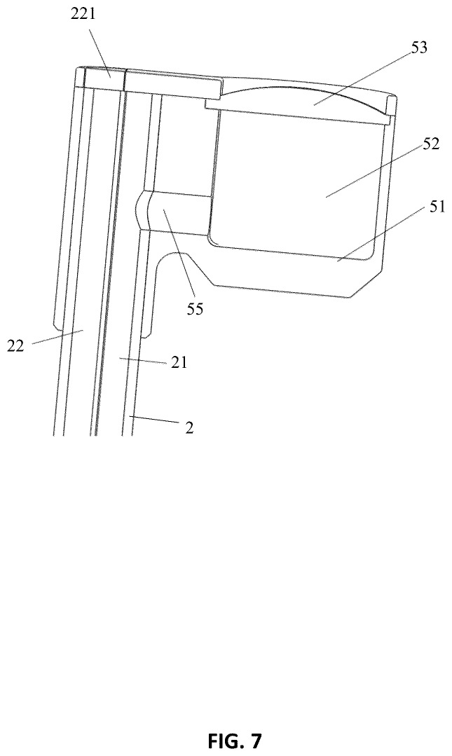

FIG. 6 is a schematic diagram illustrating a partial longitudinal cross-sectional structure of an inflation connection structure according to some embodiments of the present disclosure. FIG. 7 is a schematic diagram illustrating a partial longitudinal cross-sectional structure of an inflation connection structure with a closure head according to some embodiments of the present disclosure.

In some embodiments, as shown in FIG. 6 to FIG. 7, the inflation connection structure 3 includes an implant abutment 51 with a communicated cavity 52, the implant abutment 51 is fixedly arranged with a seal structure 53. A lumen of the connection tube 2 communicates with the communicated cavity 52. The filling structure includes a non-invasive puncture needle, the non-invasive puncture needle pierces through the seal structure 53 to communicate the communicated cavity 52.

The implant abutment 51 is configured to connect the filling structure and the isolation balloon 1. In some embodiments, the implant abutment 51 is made of a biocompatible material to ensure long-term implantation safety.

In some embodiments, the implant abutment 51 is designed in a flat or low-profile shape to reduce irritation to surrounding tissues. In some embodiments, the implant abutment 51 is fixed subcutaneously or within tissues to avoid displacement.

In some embodiments, except for the seal structure 53, which is a pierceable portion, other parts of the implant abutment 51 are made of medical-grade rigid materials, thereby facilitating fixation, ensuring the safety of the implant abutment 51, and avoiding piercing of the implant abutment 51.

The communicated cavity 52 is configured to communicate the filling structure and the connection tube 2. In some embodiments, the communicated cavity 52 has a smooth inner wall to reduce fluid resistance and avoid thrombosis or blockage.

In some embodiments, the seal structure 53 is not in communication with the exterior, thereby allowing the non-invasive puncture needle to penetrate while maintaining sealing properties.

In some embodiments, the seal structure 53 is made of an elastic material (e.g., medical silicone, polyurethane, rubber, etc.), which has self-healing properties and may withstand a plurality of punctures. In some embodiments, the seal structure 53 is made of a rubber material. In some embodiments, a thickness of the seal structure 53 is preset. For example, the thickness of the seal structure 53 is not less than 1 mm to ensure that no leakage points occur when the seal structure 53 is repeatedly pierced by the non-invasive puncture needle.

In some embodiments, the seal structure 53 is reduced in size and arranged at one side of the implant abutment 51. Compared to the seal structure 53, the connection tube 2 is arranged at another side of a bottom of the implant abutment 51 in a non-overlapping area.

In some embodiments of the present disclosure, the implant abutment 51 is provided with the communicated cavity 52 with the seal structure 53 and cooperates with the use of the non-invasive puncture needle, thereby achieving repeated inflation and deflation operations of the isolation balloon 1, and ensuring sealing performance of the tissue isolation device and reliability of long-term implantation. The self-healing property of the seal structure 53 allows the plurality of punctures without affecting sealing performance, and the special design of the non-invasive puncture needle avoids damage to the seal structure 53, thereby extending a service life of the tissue isolation device.

In some embodiments, during initial use of the tissue isolation device, an access channel for the isolation balloon 1 is first established, and the isolation balloon 1 is placed between healthy tissues and target tissues. The inflation connection structure 3 connected to the isolation balloon 1 is embedded in subcutaneous tissues. Before each irradiation treatment, skin on a surface of the inflation connection structure 3 is disinfected; the filling structure is used to penetrate the skin and the seal structure 53 to communicate the filling structure with the inflation connection structure 3, so that the isolation balloon 1 and the filling structure are connected; a filling medium in the filling structure is pushed into the isolation balloon 1 to inflate the isolation balloon 1 to a set shape to perform an isolation function. After treatment is completed, the filling medium is withdrawn from the isolation balloon 1 to deflate the isolation balloon 1; the above operations are repeated in a next treatment until a radiotherapy course is completely finished. The isolation balloon 1 is in a contracted state, a retrieval sheath is used to cover the isolation balloon 1, and the isolation balloon 1 is withdrawn from the body of the target subject by retracting the retrieval sheath, or the isolation balloon 1 in the contracted state is directly retracted. The configuration above allows repeated inflation and deflation of the isolation balloon 1 within the body of the target subject, the entire operation process is simple. Impact of the device on a patient's normal life and patient discomfort can be effectively reduced due to the implantable design, and a risk of device infection is lowered. In addition to puncture, the initial access channel for the isolation balloon 1 may also be established via endoscopy or a small incision, withdrawal of the isolation balloon 1 after use is very convenient, and there are no residues left in the body of the target subject.

In some embodiments, to prevent the non-invasive puncture needle from piercing the connection tube 2, various anti-puncture structures may be provided.

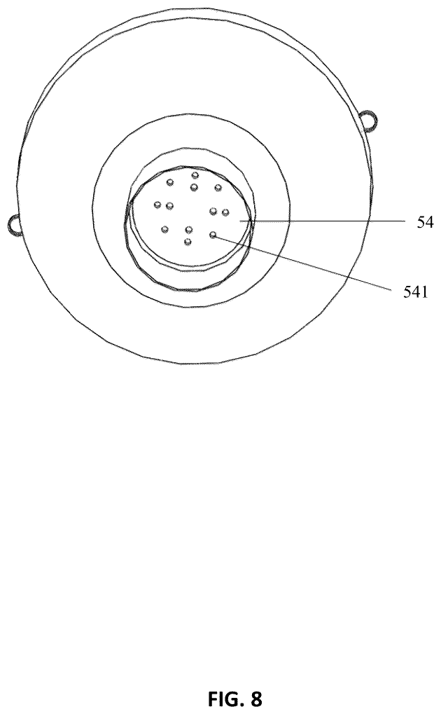

FIG. 8 is a schematic diagram illustrating a bottom plane of an implant abutment when a connection tube is arranged below the implant abutment according to some embodiments of the present disclosure.

In some embodiments, as shown in FIG. 8, a protective baffle 54 with a through hole 541 is provided at a junction between the connection tube 2 and the implant abutment 51, and a diameter of the through hole 541 is smaller than an outer diameter of the non-invasive puncture needle.

In some embodiments, the protective baffle 54 may be a thin plate made of metal or polymer material. The through hole 541 is configured to maintain a fluid channel between the implant abutment 51 and the connection tube 2. An aperture of the through hole 541 is smaller than an outer diameter of the non-invasive puncture needle, thereby preventing the non-invasive puncture needle from entering the connection tube 2, and avoiding piercing and damage to the connection tube 2.

In some embodiments, the connection tube 2 is arranged perpendicular to the implant abutment 51, an orthogonal projection of the connection tube 2 onto a first surface of the implant abutment 51 does not overlap with an orthogonal projection of the seal structure 53 onto the first surface of the implant abutment 51. The first surface is a side surface of the implant abutment 51 away from the communicated cavity 52. The perpendicular disposition means that a longitudinal axis of the connection tube 2 is perpendicular to a longitudinal axis of the implant abutment 51. The orthogonal projection refers to a projection on the first surface along a direction perpendicular to the first surface.

In some embodiments, as shown in FIG. 6 to FIG. 7, a communicated port 55 is arranged at an angle to a longitudinal axis of the communicated cavity 52, and the communicated port 55 communicates with the lumen of the connection tube 2. The communicated port 55 refers to an oblique opening connecting the communicated cavity 52 and the connection tube 2. The configuration above effectively prevents contact between the connection tube 2 and the non-invasive puncture needle, thereby minimizing the risk of piercing the connection tube 2. In some embodiments, the communicated port 55 faces perpendicular to the longitudinal axis of the communicated cavity 52 (i.e., a longitudinal axis of the communicated port 55 is perpendicular to the longitudinal axis of the connection tube 2). The communicated port 55 is arranged on a side surface of the implant abutment 51 (a surface perpendicular to the connection tube 2) and is perpendicular to a bottom of the implant abutment 51. This configuration ensures safety.

In some embodiments, a length of the non-invasive puncture needle is greater than a thickness of the seal structure 53 and is less than a sum of the thickness of the seal structure 53 and a length of the communicated cavity 52. The thickness of the seal structure 53 refers to a thickness of a sidewall of the seal structure 53 that contacts the non-invasive puncture needle. The length of the communicated cavity 52 refers to a dimension of the communicated cavity 52 along its longitudinal axis (e.g., a direction perpendicular to the connection tube 2). The configuration above effectively prevents the non-invasive puncture needle from entering the soft connection tube 2. In some embodiments, the connection tube 2 is smoothly arranged along the entire entry path, thereby effectively avoiding lumen blockage of the connection tube 2 due to bending.

In some embodiments of the present disclosure, the risk of the puncture needle mistakenly entering the connection tube 2 or piercing other parts of the implant abutment 51 can be effectively prevented through various anti-puncture configurations, thereby improving operational safety and reliability. An appropriate configuration can be selected based on usage scenarios, thereby increasing applicable scenarios of the tissue isolation device.

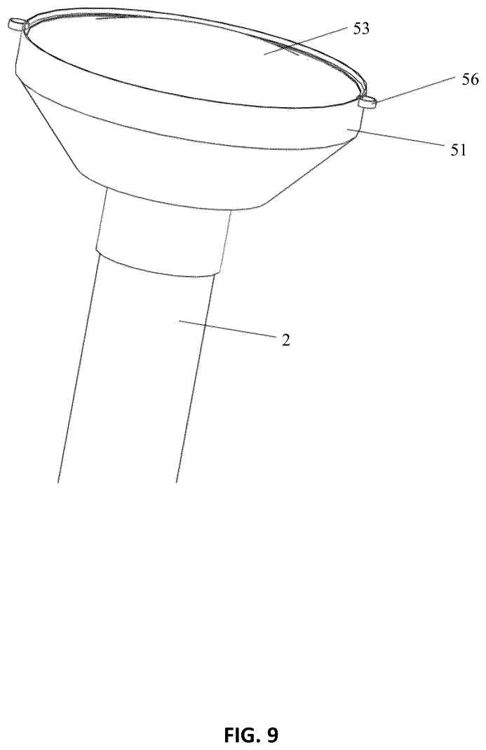

FIG. 9 is a schematic diagram illustrating a connection structure between the implant abutment and a connection tube when the connection tube is arranged below the implant abutment according to some embodiments of the present disclosure. In some embodiments, as shown in FIG. 9, the implant abutment 51 is further arranged with a suture ring 56, and the suture ring 56 secures the implant abutment 51 within subcutaneous tissues.

The suture ring 56 is configured for a suture to pass through. In some embodiments, a material of the suture ring 56 includes metal, polymer material, etc.

In some embodiments of the present disclosure, effective fixation of the implant abutment 51 can be achieved by providing a suture ring 56 on the implant abutment 51, and the risk of tissue damage or filling failure caused by movement of the implant abutment 51 can be reduced.

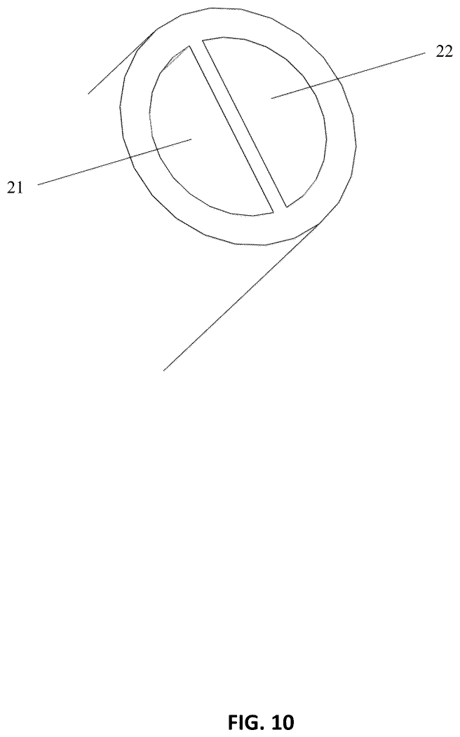

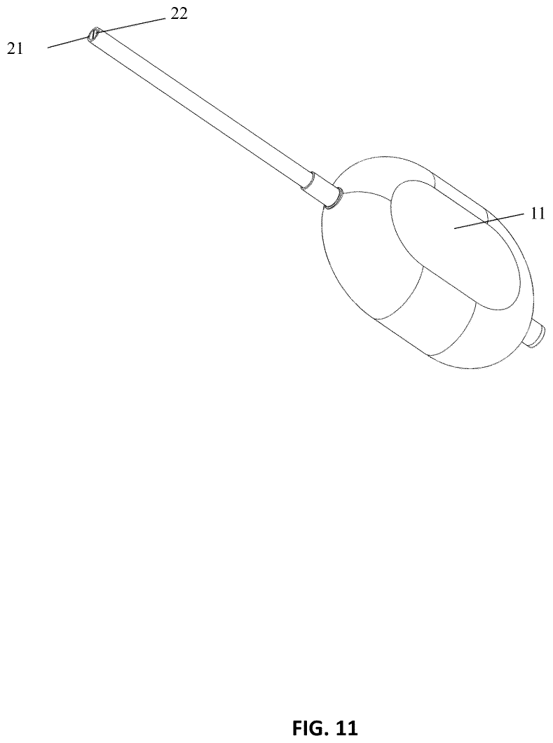

FIG. 10 is a schematic diagram illustrating a transverse cross-sectional structure of a connection tube provided with an inflation lumen and a positioning lumen according to some embodiments of the present disclosure. FIG. 11 is a schematic diagram illustrating an overall structure of a connection tube provided with the inflation lumen and a positioning lumen according to some embodiments of the present disclosure.

In some embodiments, as shown in FIG. 1 and FIG. 5 to FIG. 11, an inflation lumen 21 and a positioning lumen 22 are provided within the connection tube 2. The inflation lumen 21 is in communication with the filling structure, and a supporting and positioning rod 4 is provided within the positioning lumen 22.

The inflation lumen 21 is configured to deliver a filling medium.

In some embodiments, the material of the inflation lumen 21 may be a medical-grade polymer (e.g., polyurethane or silicone, etc.), so that the inflation lumen 21 has good sealing and flexibility.

The positioning lumen 22 is configured to accommodate the supporting and positioning rod 4.

In some embodiments, the positioning lumen 22 is designed as a rigid or semi-rigid lumen. In some embodiments, the positioning lumen 22 is be made of a wear-resistant material (e.g., PTFE or nylon, etc.).

The supporting and positioning rod 4 is configured to adjust the position and angle of the isolation balloon 1. In some embodiments, the supporting and positioning rod 4 is a rigid support rod. The supporting and positioning rod 4 may be made of materials such as stainless steel or nitinol wire. In some embodiments, an outer diameter of the supporting and positioning rod 4 matches an inner diameter of the positioning lumen 22.

In some embodiments, as shown in FIG. 6, a positioning port 222 corresponding to the positioning lumen 22 is provided on the inflation connection structure 3.

The supporting and positioning rod 4 is introduced into the entire positioning lumen 22 through the positioning port 222 on the inflation connection structure 3. When the supporting and positioning rod 4 is arranged within the positioning lumen 22, the supporting and positioning rod 4 drives the isolation balloon 1 to move or rotate within the body of a target subject to achieve positioning of the isolation balloon 1.

For example, when the isolation balloon 1 enters the body of the target subject and is placed between healthy tissues and target tissues, the supporting and positioning rod 4 is arranged within the positioning lumen 22, and a position and an angle of the supporting and positioning rod 4 are adjusted outside the body of the target subject to adjust a position and an angle of the isolation balloon 1.

In some embodiments of the present disclosure, the inflation lumen 21 and positioning lumen 22, which are independent, are arranged within the connection tube 2, so that functional separation between the filling structure and the supporting and positioning rod 4 is achieved. The inflation lumen 21 is dedicated to delivering the filling medium to ensure a stable and reliable filling process, while the supporting and positioning rod 4 within the positioning lumen 22 can precisely maintain the position and shape of the connection tube, thereby preventing it from bending or displacing during implantation or use.

In some embodiments, as shown in FIG. 6 to FIG. 7 and FIG. 10 to FIG. 11, the inflation lumen 21 and the positioning lumen 22 are arranged parallel to each other, and the inflation lumen 21 and the positioning lumen 22 are integrated into the connection tube 2. A sidewall of the inflation lumen 21 is arranged with the inflation through hole 211 communicated with the isolation balloon 1; and an end of the positioning lumen 22 that enters the body of the target subject is sealed.

In some embodiments, the isolation balloon 1 is bonded to the connection tube 2, and the inflation through hole 211 communicating with the isolation balloon 1 is only provided on the sidewall of the inflation lumen 21. The isolation balloon 1 is inflated via the inflation lumen 21 of the connection tube 2 through the inflation through hole 211.

In some embodiments, the positioning lumen 22 has no communication with the isolation balloon 1. An end of the positioning lumen 22 that first enters the body of the target subject is the first end, and the first end of the positioning lumen 22 is sealed, thereby allowing effective inflation solely through the inflation lumen 21, and preventing bodily fluids of the target subject from leaking out through the positioning lumen 22.

In some embodiments, the positioning lumen 22 is configured as lumens of different shapes. For example, the positioning lumen 22 is configured as a non-circular lumen, and the supporting and positioning rod 4 is adapted to the non-circular lumen. As another example, a cross-sectional shape of the positioning lumen 22 includes at least one straight segment. Merely by way of example, the positioning lumen 22 is configured as a square lumen, or the positioning lumen 22 is configured as a semi-circular lumen, or the positioning lumen 22 is configured as a lumen with at least one flat surface.

In some embodiments of the present disclosure, the inflation lumen 21 and the positioning lumen 22 are arranged in parallel to reduce the difficulty of lumen placement and routing.

In some embodiments, the positioning lumen 22 and the inflation lumen 21 are arranged at a side of the implant abutment 51 away from the communicated cavity 52 and are arranged perpendicular to the first surface of the implant abutment 51.

In some embodiments, as shown in FIG. 7, a closure cap 221 is provided at a rear end of the positioning lumen 22, and the closure cap 221 is used to seal the positioning lumen 22 after the supporting and positioning rod 4 is withdrawn, thereby effectively ensuring the sealed state of the connection tube 2 within the body of the target subject.

The closure cap 221 is configured to seal the positioning lumen 22. In some embodiments, the material of the closure cap 221 is medical silicone, rubber, plastic, etc., to prevent liquids, gases, or external contaminants from entering the lumen. In some embodiments, the closure cap 221 seals the positioning lumen 22 via threads, snap-fit, or press-fit structures, etc.

In some embodiments of the present disclosure, the positioning lumen 22 and the inflation lumen 21 are concentrated on the side of the implant abutment 51 away from the communicated cavity 52 and are arranged perpendicular to the first surface, thereby facilitating engagement of the supporting and positioning rod 4 with the positioning lumen 22, and avoiding interference of the supporting and positioning rod 4 with the implant abutment 51 portion during the placement process.

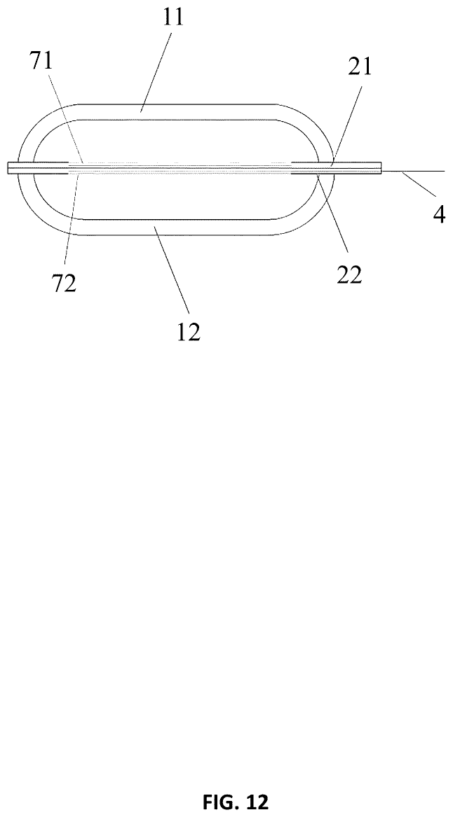

FIG. 12 is a schematic diagram illustrating a structure of a heating element and a cooling element according to some embodiments of the present disclosure. In some embodiments, as shown in FIG. 12, the tissue isolation device further includes a heating element 71 and a cooling element 72, the heating element 71 is configured to heat the first contact surface 11, and the cooling element 72 is configured to cool the second contact surface 12. The tissue isolation device further includes a first temperature sensor and a second temperature sensor, the first temperature sensor is configured to obtain temperature data of the first contact surface 11, and the second temperature sensor is configured to obtain temperature data of the second contact surface 12.

In some embodiments, the heating element 71 includes a resistance wire, a thin-film heater, or the like.

In some embodiments, the heating element 71 may also be a passive heating element, such as an ultrasonic heating resistance element (e.g., a metal wire or pad) outside the body of the target subject.

In some embodiments, the cooling element 72 includes at least one of a micro thermoelectric cooling probe based on the Peltier effect, a thermoelectric cooling wire, a micro liquid cooling circulation system, or the like. In some embodiments, the heating element 71 and the cooling element 72 are made of flexible and bendable materials.

In some embodiments, the heating element 71 and the cooling element 72 are arranged on an inner surface or an outer surface of the inflation lumen 21 and the positioning lumen 22, respectively. For example, a resistance element is arranged on the inner surface or the outer surface of the inflation lumen 21 and the positioning lumen 22. In some embodiments, the heating element 71 and the cooling element 72 are connected to a controllable power source, and the controllable power source may be disposed inside the connecting pipe or outside the body of the target subject.

In some embodiments, the heating element 71 and the cooling element 72 may also be arranged on an inner surface of the isolation balloon 1 and conform to the first contact surface 11 and the second contact surface 12. For example, during the manufacture of the tissue isolation device, the heating element 71 and the cooling element 72 are placed first, a length of the heating element 71 and a length of the cooling element 72 are both greater than a length of the isolation balloon 1. After bending the heating element 71 and the cooling element 72 into an arch shape to conform to the first contact surface 11 and the second contact surface 12, the connection tube 2 is placed.

In some embodiments, the first temperature sensor is directly attached to or embedded in the first contact surface 11 and used in conjunction with the heating element 71.

In some embodiments, the second temperature sensor is directly attached to or embedded in the second contact surface 12 and used in conjunction with the cooling element 72.

For certain tumors, local hyperthermia performed before radiotherapy can significantly enhance the therapeutic effect (sensitizing effect; heating can significantly increase the sensitivity of cancer cells to radiation). Mild cooling on the normal tissue side may reduce the metabolism and increase the tolerance to radiation (lowering temperature can slow the rate of biochemical reactions in living organisms, thereby reducing cellular metabolic activity). Therefore, the heating element 71 and the cooling element 72 can improve the therapeutic effect. The first temperature sensor and the second temperature sensor can monitor temperature changes in real time, forming a closed-loop feedback system to ensure the accuracy and reliability of temperature regulation.

In some embodiments, a thermal insulation layer is provided between the heating element 71 and the cooling element 72.

The thermal insulation layer refers to a structure used for isolating heat. In some embodiments, the thermal insulation layer is a coating of a thermal insulation material. The thermal insulation material may include aerogel, polyimide foam, etc.

In some embodiments, a separate thermal insulation layer is provided between the heating element 71 and the cooling element 72.

In some embodiments, at least one of a surface of the heating element 71 proximate to the cooling element 72 and a surface of the cooling element 72 proximate to the heating element 71 is the thermal insulation layer.

In some embodiments of the present disclosure, the thermal insulation layer is provided to reduce the influence of the temperature of the other surface on the first contact surface and the second contact surface, thereby improving the heating or cooling effect.

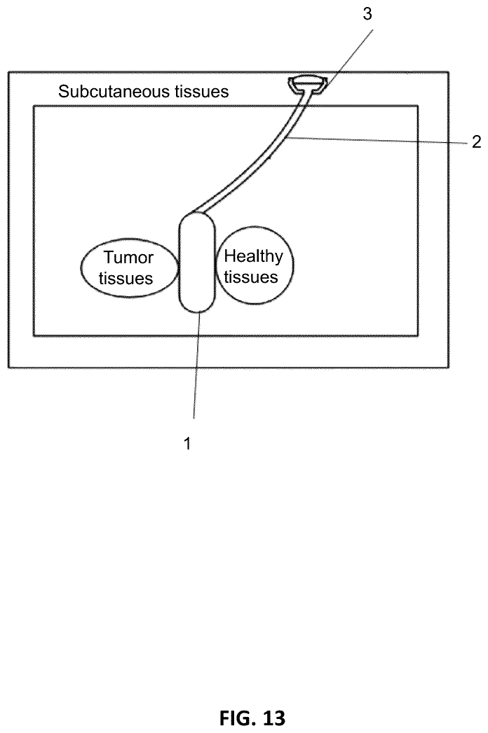

FIG. 13 is a schematic diagram illustrating a specific implementation in which an isolation balloon is arranged between deep tumor tissues and healthy tissues according to some embodiments of the present disclosure; wherein a connection tube is arranged below the implant abutment.

In some embodiments, as shown in FIG. 13, when the isolation balloon 1 is used to isolate deep tissues, the tissue isolation device is implanted via a laparoscopic surgical approach. The tissue isolation device is implanted via a laparoscopic approach, taking the isolation of pancreatic tissue receiving radiotherapy from gastric tissue as an example, implantation operations of the tissue isolation device are as follows.

First, according to a standard operative procedure for laparoscopic gastric surgery, ports are created in the abdominal wall and an access for the laparoscope and surgical instruments is established; tissues between the gastric fundus and the pancreas are isolated via laparoscopic instruments to create a space for placing the isolation balloon 1.

Second, an appropriate position on the left mid-axillary line of the upper abdomen is select to make a transverse incision with a length about 1.5-2 cm; the subcutaneous tissues are gently dissected and a small subcutaneous pocket is created; a new laparoscopic access from the site is established, and the first end of the tissue isolation device is delivered into the abdominal cavity through the new laparoscopic access, the second end corresponding to the inflation connection structure 3 is left outside the body of the target subject.

Third, the isolation balloon 1 is positioned in a space between the pancreas and the gastric fundus via the laparoscopic instruments; the inflation connection structure 3 is punctured via the non-invasive puncture needle of the filling structure and a mixture of saline and contrast agent is injected to gradually inflate the isolation balloon 1 until the preset balloon shape is achieved, and the isolation balloon is adjusted and fixed in the correct position via the laparoscopic instruments during the process.

Fourth, after the isolation balloon 1 is inflated and positioned, a laparoscopic trocar is removed from the pocket site; the inflation connection structure 3 is fixed in the subcutaneous pocket via sutures, and the skin incision is sutured.

Fifth, the other laparoscopic ports are finally removed and sutured.

Sixth, during the radiotherapy course, percutaneous puncture of the inflation connection structure 3 is performed daily to carry out an inflation operation and a deflation operation on the isolation balloon 1.

Seventh, after one radiotherapy course is completed, the mixture of saline and contrast agent is aspirated and the isolation balloon 1 is maintained under negative pressure; an original surgical incision is incised to expose the inflation connection structure 3; after removing the fixation sutures, the inflation connection structure 3 is pulled to achieve removal of the entire tissue isolation device from the body of the target subject.

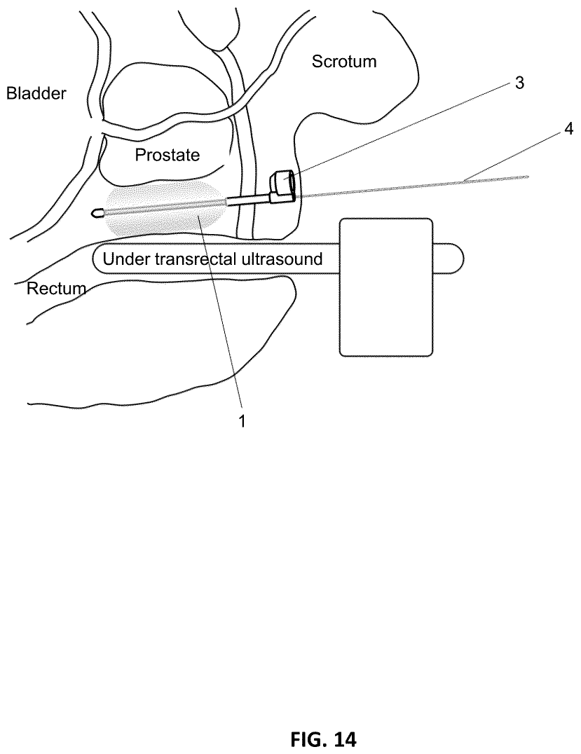

FIG. 14 is a schematic diagram illustrating a specific implementation in which an isolation balloon is arranged between prostate and rectum according to some embodiments of the present disclosure; wherein a connection tube is arranged at a side of an implant abutment and perpendicular to a bottom of the implant abutment.

In some embodiments, as shown in FIG. 14, when the isolation balloon 1 is used to isolate superficially located tissues, the tissue isolation device is implanted via a puncture approach under the guidance of CT or ultrasound. The tissue isolation device is implanted via a puncture approach. Taking the isolation of prostate cancer tissue receiving radiotherapy from intestinal tissue as an example, the implantation operations of the tissue isolation device are as follows.

First, local anesthesia is administered to the skin on the pelvic floor between the scrotum and the anus; an incision of about 1.5 cm is created, the subcutaneous tissues are gently dissected and a small pocket is created.

Second, under the guidance of transrectal ultrasound, pelvic floor tissues are penetrated via a puncture needle; a support guidewire (not shown in the figure) is deliver into Denonvilliers'aponeurosis between the prostate and the rectum via the puncture needle; an introducer sheath (not shown in the figure) is placed over the support guidewire, and a top end of the introducer sheath is delivered to a position of the prostate apex (base of the bladder). A position of the introducer sheath is confirmed and adjusted via ultrasound or CT to make the introducer sheath as coaxially as possible with a central axis of the prostate.

Third, the supporting and positioning rod 4 is placed into the positioning lumen 22; under the support and guidance of the supporting and positioning rod 4, the tissue isolation device provided by the embodiments of the present disclosure is delivered into the introducer sheath, until the first end of the tissue isolation device is flush with a first end of the introducer sheath, and then the introducer sheath is removed.

Fourth, under the support and fixation provided by the supporting and positioning rod 4, the seal structure 53 is penetrated via the non-invasive puncture needle; a mixture of saline and contrast agent is injected to gradually fill the isolation balloon 1 until a designed shape is achieved. During the process above, a position of the isolation balloon 1 is confirmed via CT, and a position and an angle of the isolation balloon 1 are adjusted using the supporting and positioning rod 4.

Fifth, after the isolation balloon 1 is filled and positioned, remove the supporting and positioning rod 4. Fix the inflation connection structure 3 with sutures in the subcutaneous tissues, and suture the skin incision.

Sixth, during the radiotherapy course, percutaneous puncture of the inflation connection structure 3 is performed daily to carry out inflation and deflation operations on the isolation balloon 1.

Seventh, after one radiotherapy course is completed, the mixture of saline and contrast agent is aspirated and the isolation balloon 1 is maintained under negative pressure. The original surgical incision is incised to expose the inflation connection structure 3. After removing the fixation sutures, the inflation connection structure 3 is pulled to achieve complete removal of the entire tissue isolation device from the body of the target subject.

In recent years, with the advancement of radiotherapy technology, the application of hypofractionated radiotherapy has become increasingly widespread. Its characteristic is a significantly shortened treatment course, typically completed within 1-2 weeks. Furthermore, with technological progress, the cycle of hypofractionated radiotherapy will become increasingly shorter. If the tissue isolation device above is applied to hypofractionated radiotherapy, implanting and removing the isolation balloon would require two skin incisions within the 1-2 week period, causing significant tissue trauma and being prone to issues such as wound infection, poor healing, and scarring. The traditional radiotherapy model typically requires a treatment cycle of 6-10 weeks. Adding the time for the patient's preoperative recovery, positioning, and radiotherapy planning, the entire radiotherapy cycle usually takes 2-3 months. The prior art CN116440428A—An implantable tissue isolation device capable of repeated inflation and deflation in vivo, has its fluid-filling component embedded subcutaneously, perfectly solving the problems of infection and device displacement risk, while the complete implantation of the isolation device has little impact on the patient's daily life. However, this technology also has two disadvantages: 1. The skin needs to be incised again when removing the balloon; 2. The fluid-filling component is located intradermally. To improve the success rate of puncture, the fluid-filling component and the resilient septum require a relatively large size (10 mm), which necessitates a larger skin incision (12-15 mm) for implantation of the complete device.

The cycle of hypofractionated radiotherapy is only 1-2 weeks. If the prior art CN116440428A is used, the implantation and removal of its isolation balloon would require repeated skin incisions within the 1-2 week period, causing significant trauma to the patient, being prone to issues such as wound infection and poor healing, and likely resulting in large scars postoperatively. Therefore, the present invention aims to provide a fluid-filling component that is partially embedded within the skin and partially exposed on the body surface, and to provide a fixing structure on the fluid-filling component. The fixing structure fixes the fluid-filling component to the peri-incisional skin and seals the incision, achieving a state where the fluid-filling component is partially embedded within the skin incision and partially exposed externally. Because the tail end of the fluid-filling component is exposed externally, the entire fluid-filling operation can be completed under direct vision, which allows for a significant reduction in the size of the resilient septum and the fluid-filling component, meaning a smaller skin incision. Simultaneously, within the 1-2 week period, provided that fixation and sealing are properly maintained, the risks of both displacement and infection of the fluid-filling component are not high, and the impact on the patient's daily life is also relatively short.

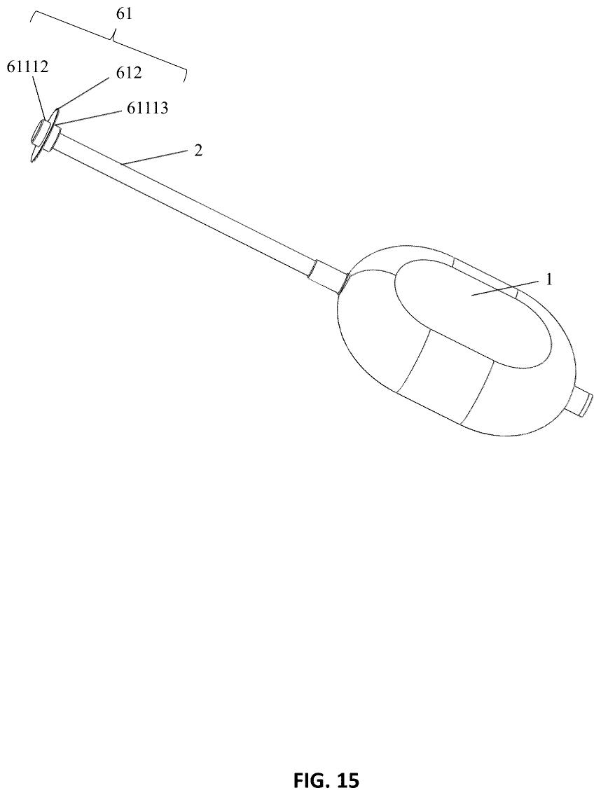

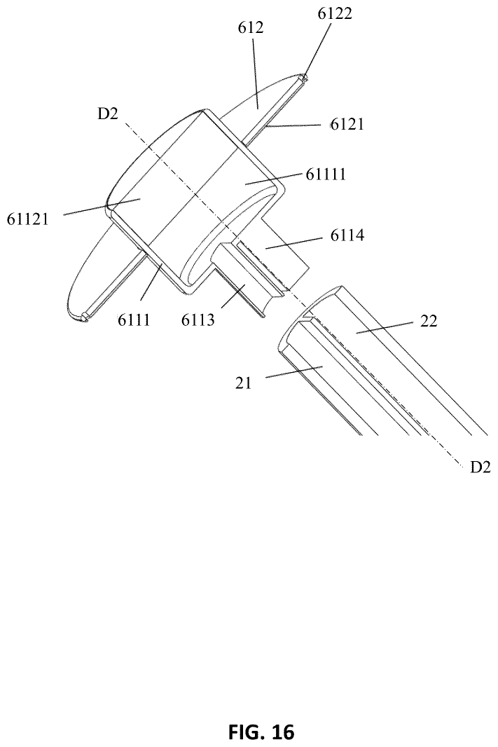

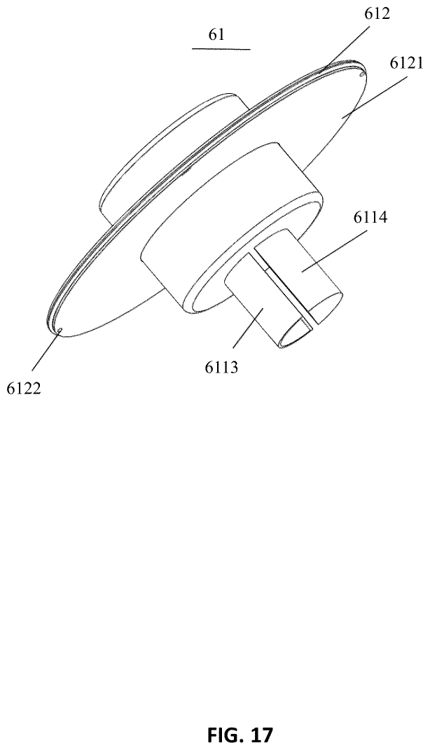

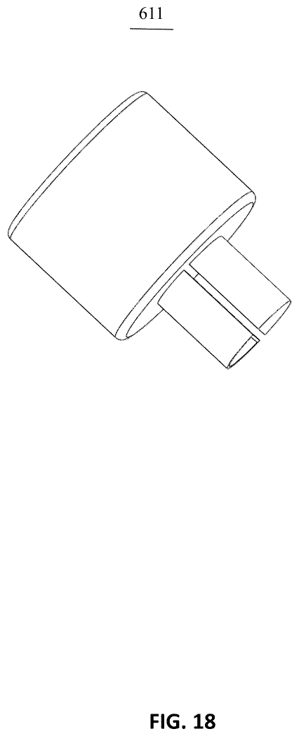

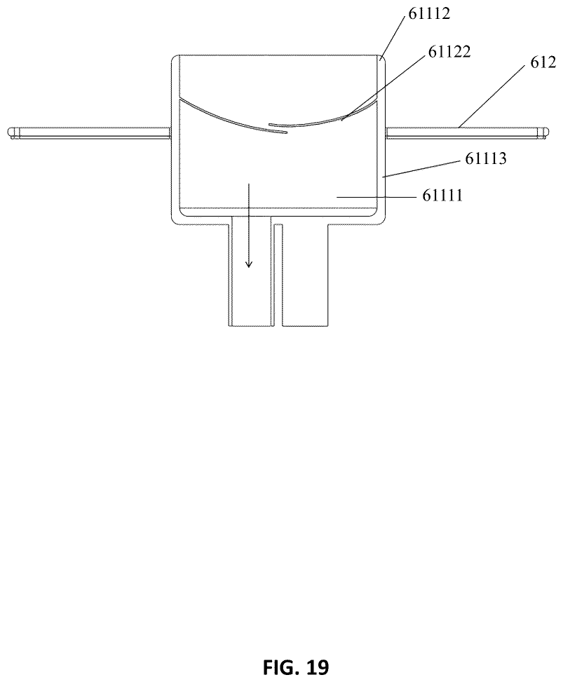

FIG. 15 is a schematic diagram illustrating an overall structure of a tissue isolation device according to some embodiments of the present disclosure. FIG. 16 is a schematic diagram illustrating a cross-sectional structure in which a portion of the connection tube and a fluid-filling component separated from the connection tube according to some embodiments of the present disclosure. FIG. 17 is a schematic diagram illustrating a structure of a fluid-filling component according to some embodiments of the present disclosure. FIG. 18 is a schematic diagram illustrating a structure of a fluid-filling structure according to other embodiments of the present disclosure. FIG. 19 is a schematic diagram illustrating a cross-sectional structure of a fluid-filling component with an occlusion valve as a sealing structure according to some embodiments of the present disclosure.

In some embodiments, as shown in FIG. 15-FIG. 19, the tissue isolation device further includes: a connection tube 2, wherein the connection tube 2 communicates with the isolation balloon 1; a fluid-filling component 61, including a fluid-filling structure 611 and a fixing structure. The fluid-filling structure 611 includes: a port body 6111, wherein an interior of the port body 6111 is arranged with a cavity 61111, the port body 6111 includes a first end and a second end arranged oppositely along a length direction of the connection tube 2, and the first end of the port body 6111 is exposed outside the body of the target subject; a sealing structure, located at the first end of the port body 6111, wherein the sealing structure is a structure of a resilient septum 61121 or an occlusion valve 61122 capable of withstanding repeated punctures; a connection head 6113, arranged at the second end of the port body 6111, wherein a through cavity is arranged inside the connection head 6113. The connection head 6113 includes a first end and a second end, the first end of the connection head 6113 communicates with the cavity 61111 of the port body 6111, the second end of the connection head 6113 communicates with the connection tube 2, and the connection head 6113 is detachable connected to the connection tube 2.

In some embodiments, the fixing structure is fixed on a skin surface of the target subject, the fixing structure is connected to an outer side of the port body 6111, the fixing structure is arranged between the first end of the port body 6111 and the second end of the port body 6111 and fixed relative to a longitudinal axis of the port body 6111, and the fixing structure divides the port body 6111 into a first portion 61112 that is exposed outside the body of the target subject and a second portion 61113 that is embedded in a skin incision.

The fluid-filling component 61 is configured to inject a filling medium into the isolation balloon 1.

The fluid-filling structure 611 is configured to store and deliver the filling medium.

The first end of the port body 6111 refers to an end of the port body 6111 that protrudes outside the body of the target subject, i.e., an end of the port body 6111 away from the isolation balloon 1. The second end of the port body 6111 refers to an end of the port body 6111 that is arranged inside the body of the target subject, i.e., an end of the port body 6111 proximate to the isolation balloon 1.

The longitudinal axis of the port body 6111 refers to a central axis of the port body 6111 along the longitudinal axis of the connection tube 2 (as shown by axis D2-D2 in FIG. 16).

The fixing structure is fixed relative to the longitudinal axis of the port body 6111, meaning the fixing structure does not undergo displacement along the longitudinal axis of the port body 6111. In some embodiments, the fixing structure may rotate about the longitudinal axis of the port body 6111. The configuration above can accommodate treatment sites with a certain degree of mobility, and the rotational movement can reduce patient injury.

In some embodiments, as shown in FIG. 16-FIG. 17, the port body 6111 is cylindrical. In some embodiments, an outer diameter of the port body 6111 ranges from 5 mm to 15 mm.

In some embodiments, the outer diameter of the port body 6111 may also be one of 5-8 mm, 8-11 mm, 11-15 mm, etc.

In some embodiments of the present disclosure, the port body 6111 is designed to be cylindrical and the outer diameter of the port body 6111 is within the range of 5-15 mm, so that a compact structure is ensured and the port body 6111 is easy to implant, thereby providing sufficient cavity volume to accommodate the required liquid or gas.

The outer diameter is not greater than a size of the incision created during the puncture process, so the skin incision size does not need to be additionally increased due to the setting of the fluid-filling component 61. The cylindrical design facilitates uniform stress distribution, reducing compression on surrounding tissues; the moderate outer diameter range ensures the mechanical strength and stability of the port body 6111, while avoiding discomfort or implantation difficulties caused by excessive size, thereby enhancing patient comfort and the practicality of the tissue isolation device while ensuring functionality.

The first portion 61112 is configured to connect with external medical devices (e.g., infusion tubes, syringes, etc.), facilitating operation by medical staff. In some embodiments, a length of the first portion 61112 of the port body 6111 is set based on clinical needs. For example, the length of the first portion 61112 is 0 mm or 5 mm. Specifically, the length should not exceed 10 mm. This setting facilitates easier operation of the port body 6111. The first portion 61112 of the port body 6111 has a small outer diameter and a small exposed length, so that even with the fluid-filling component installed, accidental contact will not significantly affect the fluid-filling component, thereby ensuring the patient's normal life and achieving an effect similar to that of an implanted device.

The second portion 61113 is connected to subcutaneous tissues or vascular access, thereby ensuring stable fixation of the port body 6111 and reducing the risk of external contamination.

The cavity 61111 is configured to accommodate a liquid or gas.

The sealing structure is configured to prevent liquid/gas leakage while allowing repeated puncture or switching operations.

The resilient septum 61121 is configured to seal the cavity 61111 of the port body 6111.

In some embodiments, the resilient septum 61121 may include a medical silicone septum, etc. In some embodiments, a thickness of the resilient septum 61121 may range from 1-5 mm. For example, the thickness of the resilient septum 61121 is set to 3 mm. The above setting can meet the requirements for repeated puncture, and also meet the sealing requirements after the resilient septum 61121 contacts the sidewall of the port body 6111. The sealing requirement is achieved by using a relatively thick membrane.

In some embodiments, the occlusion valve 61122 may include a one-way valve, a stopcock valve, etc.

In some embodiments, the occlusion valve 61122 refers to an elastic flap structure that remains in a normally open state after a syringe is inserted. When the syringe is not inserted, the occlusion valve 61122 is in a closed state to prevent liquid outflow, thereby meeting the requirements for filling and draining liquid. In some embodiments, the first portion 61112 of the port body 6111 is configured as a syringe connector for a syringe to be inserted therein. When the syringe is connected to the syringe connector, the occlusion valve 61122 becomes a normally open valve, thereby allowing direct filling/draining operations after the syringe connector is connected to the syringe. After the syringe is withdrawn, the occlusion valve 61122 prevents liquid from outflowing.

The connection head 6113 refers to an interface component arranged at the second end of the port body 6111, such as a barbed connector, a Luer connector, a quick-disconnect fluid connector, etc. In some embodiments, the second end of the connection head 6113 is in communication with the inflation lumen 21 of the connection tube 2 of the isolation balloon 1.

The through cavity refers to an internal channel of the connection head 6113, configured for the flow of liquid/gas.

The fixing structure refers to a component configured to fix the fluid-filling component 61 onto the skin surface of a target subject. In some embodiments, the fixing structure may include a medical suture anchor, an adhesive dressing, a fixation strap, etc.

Some embodiments of the present disclosure include, but are not limited to, the following beneficial effects. (1) The design of the fluid-filling component 61 enables convenient injection or withdrawal of liquid or gas, and the cavity 61111 of the port body 6111 combined with the resilient septum 61121 or the occlusion valve 61122 capable of withstanding repeated punctures ensures long-term sealing and operability. (2) The detachable connection manner of the connection head 6113 facilitates maintenance or replacement; the fixing structure stably fixes the port body 6111 onto the skin surface of the target subject, ensuring the exposed portion is easy to operate while the embedded portion remains stable, thereby improving the reliability, safety, and long-term applicability of the device. (3) The first portion 61112 of the port body 6111 is stably arranged outside the body of the target subject, and the second portion 61113 of the port body 6111 is stably embedded within the skin incision, thereby achieving the effect of avoiding repeated skin incisions within a short time for hypofractionated radiotherapy modes, and the first portion 61112 is exposed outside the body of the target subject, the outer diameter of the port body 6111 is significantly reduced, achieving the purpose of minimizing the skin incision size. The implantation and operation of the entire fluid-filling component 61 are simpler, thereby maximally meeting the needs for short-term use.

In some embodiments, as shown in FIG. 15 to FIG. 17, the fixing structure is a fixing tab 612 provided on the outer side of the port body 6111, a side of the fixing tab 612 that contacts the skin is arranged with an adhesive layer 6121, and/or a plurality of suture holes 6122 are provided on fixing tab 612.

The fixing tab 612 is configured to stably fix the tissue isolation device onto human tissue (e.g., subcutaneous fascia or muscle), preventing displacement or detachment.

In some embodiments, the fixing tab 612 is designed to be flat or sheet-like, etc. In some embodiments, the fixing tab 612 is a rigid tab or a soft tab. The rigid tab provides better fixation; the soft tab meets the movement requirements of the patient and has a high degree of conformity with the skin, thereby improving comfort degree of the patient during use.

In some embodiments, the material of the fixing tab 612 is a medical material.

The adhesive layer 6121 refers to a coating applied or attached to the surface of the fixing tab 612 that contacts the skin.

In some embodiments, the material of the adhesive layer 6121 is a biological glue, a medical pressure-sensitive adhesive, a hydrogel adhesive, etc. In some embodiments, the fixing tab 612 adheres the fluid-filling component 61 to the skin around the surgical incision via the adhesive layer 6121.

The plurality of suture holes 6122 are configured for surgical sutures to pass through. In some embodiments, the plurality of suture holes 6122 are circular holes, elliptical holes, or elongated slot holes.

In some embodiments, the fixing tab 612 may suture the fluid-filling component 61 to the skin around the surgical incision via the plurality of suture holes 6122.

In some embodiments, the fixing tab 612 is directly and fixedly arranged between the first end of the port body 6111 and the second end of the port body 6111, with no displacement or positional change relative to the longitudinal axis of the port body 6111. The connection tube 2 may be a soft and elastic tube, the shape change of the connection tube 2 can accommodate the movement requirements of the patient.

In some embodiments of the present disclosure, the fixing tab 612 is arranged on the outer side of the port body 6111, and the adhesive layer 6121 and/or the plurality of suture holes 6122 are provided on the skin-contacting surface of the fixing tab 612, so that the fixation and sealing effect of the device can be further enhanced, thereby reducing the risk of device infection and displacement. During use, the fixing structure is effectively fixed and adhered to the skin via the adhesive layer 6121 and/or the plurality of suture holes 6122, thereby achieving the purpose of fixing the fluid-filling component 61. For example, the setting of the plurality of suture holes 6122 can prevent the fluid-filling component 61 of the fluid-filling structure from detaching from the fixing structure due to external forces such as accidental contact during use.

In some embodiments, as shown in FIG. 15 to FIG. 17, the fixing tab 612 is an annular disk extending outward and surrounds the port body 6111 by 360°, and the adhesive layer 6121 is a 360° annular layer. Extending outward refers to extending in a direction away from the central axis of the port body 6111 (e.g., axis D2-D2 in FIG. 16).

In some embodiments, a protective film (not shown in the figures) is arranged on the adhesive layer 6121 before use. During use, the protective film is peeled off from the adhesive layer 6121, allowing the adhesive layer 6121 to contact the peri-incisional skin. The combination of the 360° fixing structure and the adhesive layer 6121 isolates the skin incision from the external environment, thereby avoiding infection.

The annular disk refers to a structure with a hole in the middle and a circular outer edge, configured to provide uniform fixed support.

In some embodiments of the present disclosure, the 360° annular disk fixing tab and the 360° annular adhesive layer can effectively fix the annular disk around the skin incision, thereby ensuring 100% protection of the surgical incision. After the fluid-filling component 61 is fixed, a sealing effect is achieved, preventing external bacteria from interfering with the incision; the incision is isolated from the external environment, thereby avoiding the risk of infection caused by wound exposure.

In some embodiments, a ring width of the annular disk ranges from 5 mm to 20 mm. In some embodiments, the ring width of the annular disk is one of 5-10 mm, 10-15 mm, and 15-20 mm, etc.

In some embodiments of the present disclosure, by limiting the ring width of the annular disk to the range of 5-20 mm, it is ensured that the fixing tab 612 provides sufficient supporting force while avoiding discomfort or impaired skin breathability caused by an excessively large ring width, or insufficient fixing strength caused by an excessively small ring width.

In some embodiments, as shown in FIG. 17, the plurality of suture holes 6122 are provided through an edge of the fixing tab 612, and the plurality of suture holes 6122 are uniformly distributed along a circumference of the edge of the fixing tab 612.

In some embodiments, 4 to 8 suture holes 6122 are formed through an edge of the fixing tab 612.

In some embodiments, 4 suture holes 6122 are formed through the edge of the fixing tab 612.

When using the tissue isolation device, the adhesive layer 6121 is first adhered to the peri-incisional skin, then sutures are passed through the suture holes 6122 to sew the fixing tab 612 onto the skin, thereby further reinforcing the fixation of the fixing structure. The combined arrangement of the fixing tab 612 and the adhesive layer 6121 can maximize the secure placement of the fixing tab 612 on the body surface, and the adhesive layer 6121 is maximally prevented from separating from the skin due to accidental contact during the patient's wearing of the fluid-filling component 61, thereby preventing the wound from contacting the environment and minimizing the risk of wound infection.

In some embodiments, the adhesive layer 6121 is arranged on the fixing tab 612, a plurality of suture holes 6122 are formed through an outer edge of the fixing tab 612, and the suture holes 6122 are uniformly arrayed along the edge of the fixing tab 612. During use, the adhesive layer 6121 is first adhered to the peri-incisional skin. Then, sutures are passed through the suture holes 6122 to sew the fixing tab 612 onto the skin, further reinforcing the fixation of the fixing structure. Through the above arrangement, the fixing tab 612 can be most securely placed on the body surface. This can maximally prevent the adhesive layer 6121 from separating from the skin due to accidental contact during the patient's wearing of the fluid-filling component 61, thereby preventing the wound from contacting the environment.

In some embodiments, an annular sealing patch film with a larger outer diameter is additionally applied outside the fixing structure, which is similar to common clinical reinforcement patches for dynamic glucose monitors, infusion patches, etc., thereby further enhancing fixation and sealing to maximally avoid device-related infection and displacement risks.

In some embodiments of the present disclosure, the plurality of suture holes 6122 are arranged along the edge of the fixing tab 612 uniformly and circumferentially, so that the fixing structure can be better secured, thereby allowing medical staff to flexibly choose suture positions, and ensuring the fixing tab adheres firmly to the subcutaneous tissues.

In some embodiments, an inflation lumen 21 and a positioning lumen 22 are arranged within the connection tube 2. The fluid-filling structure 611 further includes a closure head 6114, the closure head 6114 is arranged at the second end of the port body 6111 and is parallel to the connection head 6113. The closure head 6114 is a solid structure or a hollow but seal structure at both ends. The connection head 6113 extends into the inflation lumen 21, and the closure head 6114 extends into the positioning lumen 22.

More descriptions regarding the inflation lumen 21 and the positioning lumen 22 may be found in FIG. 6-FIG. 11 and the related descriptions.

The closure head refers to a structure configured to close or seal a lumen, a passage, or an interface.

In some embodiments, as shown in FIG. 17-FIG. 19, the connection head 6113 enters the inflation lumen 21 to complete the combination of the connection head 6113 and the connection tube 2, the closure head 6114 enters the positioning lumen 22 to complete the combination of the closure head 6114 and the connection tube 2, and both the connection head 6113 and the closure head 6114 are detachably connected to the connection tube 2. The detachable connection manner allows a relatively long connection tube 2 to be preset, the connection tube 2 may be cut according to the patient's condition during surgery, so that a length of the connection tube 2 precisely connects the fluid-filling component 61 and allows the fluid-filling component 61 to be embedded in the body surface, thereby avoiding problems that may arise from a fixed connection method where the length of the connection tube 2 might not match. The detachable connection manner also ensures that the supporting and positioning rod 4 can position and adjust the position of the isolation balloon 1, thereby preventing issues such as the inability to place the supporting and positioning rod 4 due to the combination method, or the need for complex structures on the fluid-filling component 611 to allow the supporting and positioning rod 4 to access the isolation balloon 1.

In some embodiments, the supporting and positioning rod 4 is inserted into the positioning lumen 22, configured to guide the isolation balloon 1 to an appropriate position and rotate to an accurate isolation angle. More descriptions regarding the supporting and positioning rod 4 may be found in the relevant content in FIG. 1 and FIG. 5.

In some embodiments, medical staff insert the supporting and positioning rod 4 through the positioning lumen 22 into the uninflated isolation balloon 1; the isolation balloon 1 sleeved on the supporting and positioning rod 4 is pushed inward through a sheath tube pre-inserted at an incision of a body surface of the patient, causing the isolation balloon 1 to move between the target tissues and the healthy tissues; and a special liquid is infused into the isolation balloon 1 through the inflation lumen 21 to inflate the balloon. The process above may be completed under ultrasound guidance, and a position and an angle of the isolation balloon 1 may be adjusted via the supporting and positioning rod 4. After positioning in place, the supporting and positioning rod 4 is withdrawn from the body of the target subject, the function of the positioning lumen 22 ceases, the positioning lumen 22 is not in communication with the inflation lumen 21 to prevent the liquid infused by the fluid-filling structure 611 of the fluid-filling component 61 into the isolation balloon 1 from entering the positioning lumen 22, thereby increasing the fluid consumption required for inflating the isolation balloon 1.

In some embodiments, the fluid-filling component 61 and the connection tube 2 are connected using a combination manner of the fluid-filling structure 611 and a catheter, which is not easily separable. In some embodiments, both the connection head 6113 and the closure head 6114 are configured as barbed connectors, thereby achieving the purpose of making it difficult to separate the fluid-filling component 61 and the connection tube 2 after combination.

In some embodiments of the present disclosure, the arrangement of the closure head 6114 can prevent liquid from entering the positioning lumen 22. The combined stability of the entire fluid-filling component 61 and the connection tube 2 after combination is maintained, thereby preventing gaps from forming between the port body 6111 and the positioning lumen 22 due to skewing, which could allow liquid to enter the positioning lumen 22, thereby avoiding continuous stimulation to the wound caused by movement to prevent increased pain for the patient.

In some embodiments, as shown in FIG. 16, the outer diameter of the port body 6111 is greater than or equal to an outer diameter of the connection tube 2. The fixing tab 612 is oriented perpendicular to the longitudinal axis of the port body 6111. A length of the first portion 61112 of the port body 6111 is less than or equal to 10 mm.

In some embodiments, the outer diameter of the port body 6111 ranges from 5 mm to 15 mm. For example, the outer diameter of the port body 6111 is 5 mm. The arrangement above corresponds very well to a size of the puncture sheath, the puncture incision created by the puncture sheath directly accommodates the embedding of the port body 6111, thereby eliminating the need to enlarge the skin incision again and significantly reducing the size of the skin incision. Due to the embedding manner, it is only necessary to withdraw the liquid from the isolation balloon 1 and separate the fixing tab 612 from the skin to withdraw the isolation balloon 1 from the body of the target subject when the procedure is finished; and the skin incision is sutured. The entire process involves only one skin incision and suturing operation, causing minimal impact on the patient.

In some embodiments of the present disclosure, the outer diameter of the port body 6111 is designed to be greater than or equal to the outer diameter of the connection tube 2, so that the mechanical strength of the connection portion is ensured, thereby avoiding structural damage due to stress concentration and enhancing sealing reliability. The fixing tab 612 is arranged perpendicular to the longitudinal axis of the port body 6111, thereby providing a stable supporting surface for easy fixation on the skin surface and reducing the risk of device displacement. The length of the first portion 61112 of the port body 6111 is controlled within 10 mm, thereby ensuring sufficient operating space while avoiding discomfort or implantation difficulty caused by excessive length, and improving patient comfort and long-term use stability while ensuring device functionality.

In some embodiments, when using the tissue isolation device, a puncture instrument is first used to perform puncture through a small incision on the body surface, creating a channel from the body surface to the tissue to be isolated, and a guide sheath is inserted. After inserting the supporting and positioning rod 4 into the positioning lumen 22, the isolation balloon 1 is guided into the space between the target tissues and the healthy tissues through the guide sheath under the support and guidance of the supporting and positioning rod 4. After removing the guide sheath, a special liquid is infused into the isolation balloon 1 through the inflation lumen 21 to inflate the balloon; the position and the angle of the isolation balloon 1 are adjusted via the supporting and positioning rod 4 until satisfactory, and then the supporting and positioning rod 4 is removed. The liquid within the isolation balloon 1 is withdrawn, or the connection tube 2 is manually clamped shut, and the length of the connection tube 2 is cut. The fluid-filling component 61 is combined with the cut connection tube 2, ensuring that the connection head 6113 is inserted into the inflation lumen 21 and the closure head 6114 is inserted into the positioning lumen 22. After combination, the second portion 61113 of the port body 6111 is embedded within the skin incision; the protective film on the adhesive layer 6121 of the fixing tab 612 is peeled off, allowing the adhesive layer to closely adhere to the peri-incisional skin; sutures are used to pass through the suture holes 6122 on the fixing tab 612 for further suturing reinforcement, causing the entire fixing structure to be fixed to the peri-incisional skin, while simultaneously achieving sealing of the skin incision, thereby completing the setup of the tissue isolation device. When radiotherapy is required, a puncture operation is performed on the resilient septum 61121 using a puncture needle; the puncture needle is connected to a syringe, and liquid is injected into the isolation balloon 1 via the syringe, isolating the target tissues from the healthy tissues. After the isolation balloon 1 is inflated, the puncture needle is separated from the resilient septum 61121; after a single radiotherapy session is completed, the puncture needle is again inserted into the sealing structure combination, and the liquid within the isolation balloon 1 is withdrawn. The above operations are repeated for subsequent radiotherapy sessions, after the 1-2 week hypofractionated radiotherapy course is completed, the isolation balloon 1 is first completely deflated; the adhesive layer 6121 is separated from the skin, and the sutures in the suture holes 6122 are removed, causing the fixing tab 612 to separate from the skin; the fluid-filling structure 611 along with the isolation balloon 1 are withdrawn from the body of the target subject by pulling.

In some embodiments, for isolation balloons 62 in relatively spacious tissues, inflation and deflation may not be required for each radiotherapy session, and the isolation balloons 62 is capable of remaining in the inflated state continuously. When the sealing structure is the occlusion valve 61122, it is only necessary to insert the syringe into the syringe connector of the first portion 61112 of the port body 6111 to perform the liquid infusion/withdrawal operation; other operations are the same.

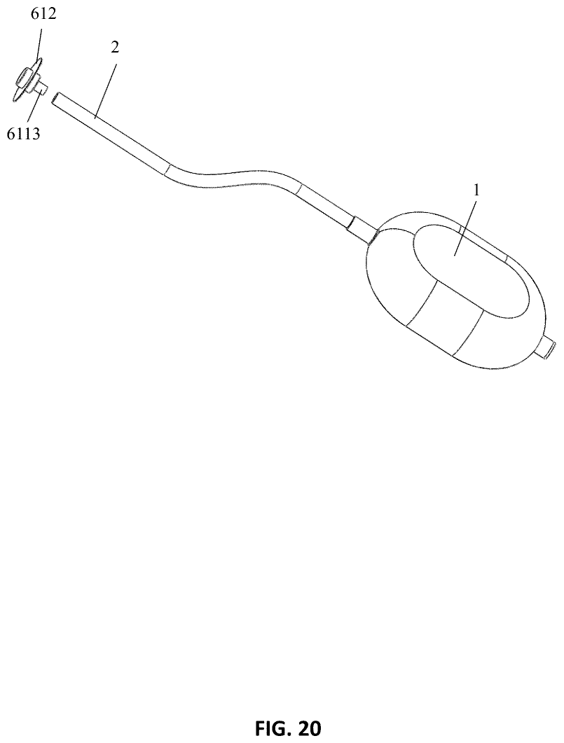

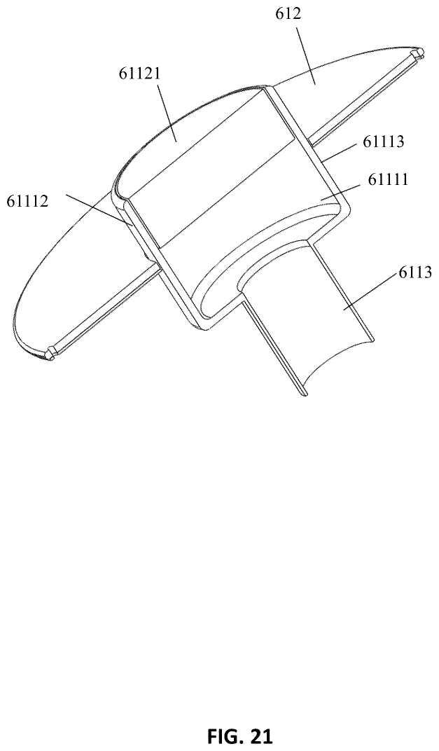

FIG. 20 is a schematic diagram illustrating a structure in which a fluid-filling component is separated from an isolation balloon according to some embodiments of the present disclosure. FIG. 21 is a schematic diagram illustrating a cross-sectional structure of a fluid-filling component according to some embodiments of the present disclosure.

In some embodiments, as shown in FIG. 20-FIG. 21, the connection tube 2 is provided with only one inflation lumen 21, the second end of the port body 6111 is provided only with the connection head 6113, the positioning lumen 22 and the corresponding closure head 6114 are not arranged. Other arrangements are the same as the relevant arrangements in FIG. 15-FIG. 18, which are not repeated here.

In some embodiments, when using the tissue isolation device, there is no need to use the supporting and positioning rod 4 for positioning the position and angle of the isolation balloon 1. Instead, under direct endoscopic vision, the isolation balloon 1 is introduced into the body of the target subject through a small body surface incision using the endoscopic surgical instruments. The isolation balloon 1 is inflated and fixed after being placed at the position requiring isolation; the connection tube 2 is cut to a suitable length and connected to the fluid-filling component 61; other operational steps are the same as the operational steps of the tissue isolation device described previously and are not repeated here.

In some embodiments, a pressure sensor is further arranged within the isolation balloon 1, the pressure sensor is configured to obtain a balloon pressure. The tissue isolation device further includes a processor, and the processor is configured to: determine a fine-tuning infusion amount and a pressure warning based on the balloon pressure and infusion information during a radiotherapy process; control the filling structure to perform infusion on the isolation balloon based on the fine-tuning infusion amount.

The pressure sensor refers to a device for measuring an internal pressure of the isolation balloon. In some embodiments, the pressure sensor is arranged inside the isolation balloon.

In some embodiments, the pressure sensor may be a pressure sensor array to obtain balloon pressures at a plurality of positions.

The balloon pressure refers to a pressure exerted by a fluid (e.g., a liquid or a gas) inside the isolation balloon 1 on a wall of the balloon. The balloon pressure may include pressure magnitudes and pressure directions at a plurality of positions inside the isolation balloon 1.

In some embodiments, the processor is configured to process information and/or data related to the tissue isolation device. In some embodiments, the processor may be communicatively connected to the pressure sensor, the filling structure, etc.

In some embodiments, the processor may include one or more of a microcontroller (MCU), an embedded processor, a graphics processing unit (GPU), or a combination thereof. The processor communicates and connects with the filling structure.

The infusion information refers to data related to an inflation process of the isolation balloon.

In some embodiments, the infusion information may include a current inflation volume, an inflation rate, etc. For example, the infusion information may be a cumulative volume of normal saline and a contrast agent that have been inflated into the isolation balloon.

The fine-tuning infusion amount refers to a fine-tuning amount required for pressure relief or inflation. The fine-tuning infusion amount further includes a time point at which fine-tuning needs to be performed.

The pressure warning refers to a warning issued when an abnormal condition occurs in the balloon pressure.

In some embodiments, the processor may construct a first vector to be matched based on the balloon pressure and the infusion information. The processor may retrieve a first vector database based on the first vector to be matched, and obtain a first reference vector whose vector distance from the first vector to be matched is less than a distance threshold. The processor may determine a historical fine-tuning infusion amount and a historical pressure warning corresponding to the first reference vector as the fine-tuning infusion amount and the pressure warning.

In some embodiments, the first vector database stores a plurality of first reference vectors and the corresponding historical fine-tuning infusion amounts and historical pressure warnings. The first reference vector is constructed based on a historical balloon pressure and historical infusion information. The distance threshold may be preset based on experience.

In some embodiments, the processor may dynamically adjust the fine-tuning infusion amount and the pressure warning, and update the first vector database by comparing a real-time balloon pressure-volume (P-V) curve with a preset P-V standard curve.

In some embodiments, abnormal conditions that trigger the pressure warning may include abnormal pressure increase, abnormal pressure stability or decrease, abnormal pressure fluctuation, etc. The abnormal pressure increase indicates that the isolation balloon 1 may be stuck, restricted by a narrow area, or encountering hardened tissues, resulting in shape distortion. The abnormal pressure stability or decrease indicates that the isolation balloon 1 may have a leak or rupture. The abnormal pressure fluctuation indicates that the isolation balloon 1 may be subjected to abnormal compression from surrounding tissues (e.g., a tumor) or a significant change in a patient's posture.

In some embodiments, if the processor does not find a historical pressure warning by retrieving the first vector database, it indicates that no abnormal condition exists.

In some embodiments, the processor may, in response to existence of the pressure warning, control the filling structure to stop inflating the isolation balloon 1. The processor may, in response to absence of the pressure warning, control the filling structure to continue inflation after adjusting the infusion information based on the fine-tuning infusion amount.

In some embodiments of the present disclosure, by monitoring the balloon pressure in real time and combining the infusion information, the processor dynamically determines the fine-tuning infusion amount and automatically adjusts the inflation volume inside the balloon, thereby maintaining a stable isolation state and a precise isolation distance.

In some embodiments, a displacement sensor is disposed on the portion of the connection tube 2 located inside the isolation balloon 1. The processor is configured to: acquire an initial position of the isolation balloon 1 via the displacement sensor; obtain a current position of the isolation balloon 1 via the displacement sensor at a preset interval; determine a displacement of the isolation balloon 1 based on the initial position and the current position, and send a movement alert to a terminal device in response to the displacement of the isolation balloon 1 exceeding a preset threshold. The operator may use the movement alert received by the terminal device to check the position of the isolation balloon 1 via ultrasound or CT.

The displacement sensor refers to a sensor used to detect a displacement situation of the isolation balloon 1. In some embodiments, the displacement sensor may be an electromagnetic sensor (EM sensor), a micro inertial measurement unit (IMU), or the like.

The initial position refers to a position of the isolation balloon 1 in an initial state, such as a position after the isolation balloon 1 being implanted in the body. The current position refers to the position of the isolation balloon 1 at the present moment. Merely by way of example, the current position and initial position may be represented by coordinates. The displacement of the isolation balloon 1 refers to a movement distance of the isolation balloon 1 relative to the initial position. In some embodiments, the processor may take an absolute value of a vector distance (e.g., cosine distance) between the current position and the initial position as the displacement.

The preset threshold refers to a critical displacement value used to determine abnormal movement of the isolation balloon 1. The preset threshold may be set empirically.

The movement alert refers to a warning message indicating abnormal movement of the isolation balloon 1.

The terminal device may be a mobile phone, personal computer, tablet, or the like. The processor is communicatively connected to the terminal device.

In some embodiments, the processor is further configured to: determine a first target temperature of the heating element 71 and a second target temperature of the cooling element 72 based on the balloon pressure, a protective agent characteristic, a preset radiotherapy plan, and the infusion information.

The protective agent characteristic refers to a related attribute of the radioprotective agent. For example, the protective agent characteristic includes a concentration of the radioprotective agent, an optimal temperature, etc. In some embodiments, the protective agent characteristic may be preset by a technician.

The preset radiotherapy plan refers to a preset radiotherapy parameter. For example, the preset radiotherapy plan includes a time point, a dose, a geometric shape of a target area, a desired isolation distance, etc., for each radiotherapy session. In some embodiments, the preset radiotherapy plan may be preset by a technician.

The first target temperature refers to a temperature that the heating element 71 needs to reach before performing radiotherapy. The second target temperature refers to a temperature that the cooling element 72 needs to reach before performing radiotherapy.

In some embodiments, the first target temperature and the second target temperature may be temperature sequences, including the first target temperature and the second target temperature corresponding to a plurality of time points.

In some embodiments, the processor may determine the first target temperature of the heating element 71 and the second target temperature of the cooling element 72 through a temperature model based on the balloon pressure, the protective agent characteristic, the preset radiotherapy plan, and the infusion information.

The temperature model refers to a model for determining the target temperature. In some embodiments, the temperature model may be a machine learning model. For example, the temperature model may include any one or a combination of a Convolutional Neural Network (CNN) or other custom model structures. In some embodiments, an input of the temperature model may include the balloon pressure, the radioprotective agent characteristic, the preset radiotherapy plan, and the infusion information, and an output may include the first target temperature of the heating element 71 and the second target temperature of the cooling element 72.

In some embodiments, the temperature model may be obtained through training based on a plurality of first training samples with first labels. The first training samples may include sample balloon pressures, sample radioprotective agent characteristics, sample preset radiotherapy plans, and sample infusion information. The first labels may include actual temperatures of heating elements and actual temperatures of cooling elements corresponding to the first training samples.

In some embodiments, the first training samples and the first labels may be obtained through experimental data.

In some embodiments, the processor may perform training using various methods based on the first training samples and the first labels. For example, training may be performed based on a gradient descent method. Merely by way of example, the processor may input a plurality of first training samples with first labels into an initial temperature model, construct a loss function based on the first labels and results of the initial temperature model, and iteratively update parameters of the initial temperature model based on the loss function. When the loss function of the initial temperature model satisfies a preset condition, model training is completed, and a trained temperature model is obtained. The preset condition may include convergence of the loss function, a number of iterations reaching a threshold, etc.

In some embodiments, the processor may control the heating element 71 to heat based on the first target temperature, and control the cooling element 72 to cool based on the second target temperature.

The basic concepts have been described above. Obviously, to a person skilled in the art, the above detailed disclosure is merely an example and does not constitute a limitation to the present disclosure. Although not explicitly stated herein, a person skilled in the art may make various modifications, improvements, and amendments to the present disclosure. Such modifications, improvements, and amendments are suggested in the present disclosure, so they still fall within the spirit and scope of the exemplary embodiments of the present disclosure.

It should be emphasized and noted that “an embodiment”, “one embodiment”, or “an alternative embodiment” mentioned two or more times at different locations in the present disclosure does not necessarily refer to the same embodiment. In addition, certain features, structures, or characteristics in one or more embodiments of the present disclosure may be appropriately combined.

Similarly, it should be noted that, to simplify the expressions disclosed in the present disclosure to help understand one or more inventive embodiments, a plurality of features are sometimes grouped into one embodiment, drawing, or description thereof in the foregoing description of the embodiments of the present disclosure. However, this disclosure method does not mean that the object of the present disclosure requires more features than those mentioned in the claims. Rather, claimed subject matter may lie in less than all features of a single foregoing disclosed embodiment.

In some embodiments, numbers describing quantities of components and attributes are used. It should be understood that such numbers used in the description of the embodiments are modified by the modifiers “approximately”, “about”, or “substantially” in some examples. Unless otherwise stated, “approximately”, “about”, or “substantially” indicates that the number allows a variation of ±20%. Accordingly, in some embodiments, numerical parameters used in the specification and claims are approximate values, which may vary according to the characteristics required by individual embodiments. In some embodiments, numerical parameters should consider specified significant digits and adopt a general digit retention method. Although numerical ranges and parameters used to confirm the breadth of the scope in some embodiments of the present disclosure are approximate values, such numerical values are set as accurately as possible within a feasible range in specific embodiments.

For each patent, patent application, patent application publication, and other materials, such as articles, books, specifications, publications, documents, etc., cited in the present disclosure, the entire contents thereof are hereby incorporated into the present disclosure by reference. Except for application history documents that are inconsistent with or conflict with the content of the present disclosure, documents that limit the broadest scope of the claims of the present disclosure (currently or subsequently attached to the present disclosure) are also excluded. It should be noted that if the description, definition, and/or use of terms in the accompanying materials of the present disclosure are inconsistent with or conflict with those in the present disclosure, the description, definition, and/or use of terms in the present disclosure shall prevail.

Finally, it should be understood that the embodiments described in the present disclosure are merely illustrative of the principles of the embodiments of the present disclosure. Other modifications may also fall within the scope of the present disclosure. Accordingly, as an example and not by way of limitation, alternative configurations of the embodiments of the present disclosure may be considered consistent with the teachings of the present disclosure. Accordingly, the embodiments of the present disclosure are not limited to the embodiments explicitly introduced and described in the present disclosure.

What is claimed is:

1. A tissue isolation device capable of repeated inflation and deflation in vivo, comprising:
an isolation balloon, wherein the isolation balloon is arranged between healthy tissues and target tissues for a radiotherapy course, and the isolation balloon is removed from a body of a target subject following the radiotherapy course; the isolation balloon is operable to assume an inflated state and a deflated state, the isolation balloon in the inflated state is used to isolate the healthy tissues and the target tissues, and the isolation balloon in the deflated state is used to maintain a physiological state of the healthy tissues and the target tissues;
the isolation balloon includes a first contact surface and a second contact surface, in the inflated state, the first contact surface contacts the healthy tissues, and the second contact surface contacts the target tissues; and
when the isolation balloon is in the inflated state, a minimum distance between the first contact surface and the second contact surface is greater than or equal to a distance required for a radiation dose to fall off to within a tolerance threshold of the healthy tissues, a maximum distance between the first contact surface and the second contact surface is less than or equal to a maximum tolerable distraction distance of the healthy tissues.

2. The tissue isolation device of claim 1, wherein the tissue isolation device further includes:
an inflation connection structure, configured to connect the isolation balloon to a filling structure located outside the body of the target subject; wherein the inflation connection structure is implanted in subcutaneous tissues of the target subject, and the inflation connection structure and the filling structure are connected to control inflation and deflation of the isolation balloon.

3. The tissue isolation device of claim 1, wherein the tissue isolation device further includes:
a connection tube, configured to communicate the inflation connection structure to the isolation balloon, wherein an end of the connection tube that first enters the body of the target subject is designated as a first end, and the first end of the connection tube is arranged at least partially into an interior of the isolation balloon; and the connection tube is arranged with an inflation through hole communicated with the isolation balloon.

4. The tissue isolation device of claim 3, wherein the inflation connection structure includes an implant abutment with a communicated cavity, the implant abutment is fixedly arranged with a seal structure; a lumen of the connection tube communicates with the communicated cavity; the filling structure includes a non-invasive puncture needle, the non-invasive puncture needle pierces through the seal structure to communicate the communicated cavity.

5. The tissue isolation device of claim 4, wherein
a protective baffle with a through hole is provided at a junction between the connection tube and the implant abutment, and a diameter of the through hole is smaller than an outer diameter of the non-invasive puncture needle;
or, the connection tube is arranged perpendicular to the implant abutment, an orthogonal projection of the connection tube onto a first surface of the implant abutment does not overlap with an orthogonal projection of the seal structure onto the first surface of the implant abutment, the first surface is a side surface of the implant abutment away from the communicated cavity;
or, a communicated port is arranged at an angle to a longitudinal axis of the communicated cavity, and the communicated port communicates with the lumen of the connection tube;
or, a length of the non-invasive puncture needle is greater than a thickness of the seal structure and is less than a sum of the thickness of the seal structure and a length of the communicated cavity.

6. The tissue isolation device of claim 5, wherein the implant abutment is further arranged with a suture ring, and the suture ring secures the implant abutment within subcutaneous tissues.

7. The tissue isolation device of claim 1, wherein outer sides or peripheral regions of the first contact surface and the second contact surface are higher than the first contact surface and the second contact surface; or, on a surface of the isolation balloon, the outer sides or the peripheral regions of the first contact surface and the second contact surface are lower than the first contact surface and the second contact surface, and the peripheral regions of the first contact surface and the second contact surface blend through a radial contour.

8. The tissue isolation device of claim 7, wherein in the inflated state, a distance between the first contact surface and the second contact surface ranges from 1 cm to 2 cm.

9. The tissue isolation device of claim 1, wherein the isolation balloon is coated with a drug-carrying coating, and the first contact surface is loaded with a radioprotective agent and/or a slow-release local anesthetic; the second contact surface is loaded with a chemotherapeutic agent and/or the slow-release local anesthetic.

10. The tissue isolation device of claim 3, wherein the connection tube is provided with an inflation lumen and a positioning lumen; the inflation lumen communicates with the filling structure, and a supporting and positioning rod is arranged within the positioning lumen.

11. The tissue isolation device of claim 10, wherein the positioning lumen and the inflation lumen are arranged parallel to each other, and the positioning lumen and the inflation lumen are integrated into the connection tube;
a sidewall of the inflation lumen is arranged with the inflation through hole communicated with the isolation balloon; and
an end of the positioning lumen that enters the body of the target subject is sealed.

12. The tissue isolation device of claim 10, wherein the positioning lumen and the inflation lumen are arranged at a side of the implant abutment away from the communicated cavity and are arranged perpendicular to the first surface of the implant abutment.

13. The tissue isolation device of claim 1, wherein the tissue isolation device further comprising:
a connection tube, wherein the connection tube communicates with the isolation balloon;
a fluid-filling component, including a fluid-filling structure and a fixing structure, wherein
the fluid-filling structure includes:
a port body, wherein an interior of the port body is arranged with a cavity, the port body includes a first end and a second end arranged oppositely along a length direction of the connection tube, and the first end of the port body is exposed outside the body of the target subject;
a sealing structure, located at the first end of the port body, wherein the sealing structure is a structure of a resilient septum or an occlusion valve capable of withstanding repeated punctures;
a connection head, arranged at the second end of the port body, wherein a through cavity is arranged inside the connection head, the connection head includes a first end and a second end, the first end of the connection head communicates with the cavity of the port body, the second end of the connection head communicates with the connection tube, and the connection head is detachable connected to the connection tube;
wherein the fixing structure is fixed to a skin surface of the target subject, the fixing structure is connected to an outer side of the port body, the fixing structure is arranged between the first end of the port body and the second end of the port body and fixed relative to a longitudinal axis of the port body, and the fixing structure divides the port body into a first portion that is exposed outside the body of the target subject and a second portion that is embedded in a skin incision.

14. The tissue isolation device of claim 13, wherein the fixing structure is a fixing tab provided on the outer side of the port body, a side of the fixing tab that contacts with skin is arranged with an adhesive layer, and/or a plurality of suture holes are provided on the fixing tab.

15. The tissue isolation device of claim 14, wherein the fixing tab is an annular disk that extends outward and surrounds the port body by 360 degrees, and the adhesive layer is a 360-degree annular layer.

16. The tissue isolation device of claim 15, wherein a ring width of the annular disk ranges from 5 mm to 20 mm.

17. The tissue isolation device of claim 14, wherein the plurality of suture holes are provided through an edge of the fixing tab, and the plurality of suture holes are uniformly distributed along a circumference of the edge of the fixing tab.

18. The tissue isolation device of claim 13, wherein a positioning lumen and an inflation lumen are arranged within the connection tube, the fluid-filling structure further includes a closure head, the closure head is arranged at the second end of the port body and is parallel to the connection head; and the closure head is a solid structure or a hollow but seal structure at both ends; and
the connection head extends into the inflation lumen, and the closure head is inserted into the positioning lumen.

19. The tissue isolation device of claim 13, wherein the port body is cylindrical, and an outer diameter of the port body ranges from 5 mm to 15 mm.

20. The tissue isolation device of claim 19, wherein the outer diameter of the port body is greater than or equal to an outer diameter of the connection tube; the fixing tab is oriented perpendicular to the longitudinal axis of the port body, and a length of the first portion of the port body is less than or equal to 10 mm.
